(19) United States
(12) Patent Application Publication
(10) Pub. No.: US 2026/0185168 A1
(43) Pub. Date: Jul. 2, 2026

(54) NEUTRAL SNAPSHOT MARKER OF PISUM SATIVUM L. AND USE THEREOF IN ANALYSIS OF POPULATION GENETIC DIVERSITY

(71) Applicants: Shandong Academy of Agricultural Sciences, Jinan (CN); Institute of Crop Science, Chinese Academy of Agricultural Sciences, Beijing (CN)

(72) Inventors: Hanfeng Ding, Jinan (CN); Xuxiao Zong, Jinan (CN); Dong Wang, Jinan (CN); Tao Yang, Jinan (CN); Nana Li, Jinan (CN); Rong Liu, Jinan (CN); Xiaoyan Zhang, Jinan (CN)

(21) Appl. No.: 18/927,738

(22) Filed: Oct. 25, 2024

Related U.S. Application Data
(63) Continuation of application No. PCT/CN2023/073449, filed on Jan. 21, 2023.

(30) Foreign Application Priority Data
Apr. 25, 2022 (CN) 202210439681.6

(51) Int. Cl. C12Q 1/6895; G16B 20/40

(52) U.S. Cl. CPC C12Q 1/6895; G16B 20/40; C12Q 2600/13; C12Q 2600/156

(57) ABSTRACT

The present invention discloses a set of neutral SNaPshot markers of Pisum sativum L. and use thereof in analysis of population genetic diversity. In the present invention, 432 accessions of Pisum sativum L. germplasms are used as test materials, and subjected to analysis of genetic diversity and population genetic structure of Pisum sativum L. through a set of neutral SNaPshot markers (46 neutral markers) of Pisum sativum L. The neutral markers can better group the Pisum sativum L. germplasms according to their geographic origins due to their independence of functional genes such as heat tolerance, and are more consistent with a type of sowing date. Experiments show that the neutral markers of the present invention are scientifically selected and evenly distributed on chromosomes.

### FIELD OF TECHNOLOGY

The present invention relates to a set of neutral SNaPshot markers of Pisum sativum L. and use thereof in analysis of population genetic diversity, belonging to the technical field of plant genetics.

### BACKGROUND

Pisum sativum L. (2n=14) is a cold-season edible legume crop widely planted in temperate regions. It is rich in nutritional value and is an important source of protein, starch, sugar, crude fiber, vitamins and low fat. Meanwhile, rhizobia in the root system of Pisum sativum L. can fix nitrogen in the atmosphere, so that soil fertility is increased and environmental pollution is reduced. Pisum sativum L. is an excellent crop for multiple purposes of grain, vegetable, feed and fertilizer. According to the statistics of the Food and Agriculture Organization of the United Nations (FAO), in 2019, the total output of edible legumes in the world was 88,379,804 tons, with the output of Pisum sativum L. ranked only second to that of common kidney beans, and Pisum sativum L. being the second largest edible legume crop. A total of 98 countries and regions in the world produce dried grains of Pisum sativum L., with a total output of 1,4184,249 tons. The top three countries are Canada, the Russian Federation and China, respectively. In the same year, a total of 87 countries and regions in the world produce green grains of Pisum sativum L., with a total output of 21,766,060 tons, and the top three countries are China, India and France, respectively. The output of dried grains of Pisum sativum L. in China ranks third in the world, and the output of green grains of Pisum sativum L. in China ranks first in the world. It is undoubtedly that China is the largest producer of Pisum sativum L. in the world, but it still needs to import a large amount of Pisum sativum L. from Canada and the like countries every year to meet the ever increasing consumption demand. Therefore, it is of great importance to understand the genetic diversity and population genetic relationship among each accession of Pisum sativum L. germplasms for the research on genetic improvement and selection of suitable parents in breeding of Pisum sativum L.

In recent years, with the rise of single nucleotide polymorphism markers (SNPs), they were widely applied in many research fields of Pisum sativum L., such as association mapping, genome-wide association study (GWAS), QTL identification, candidate gene mining, genetic linkage map construction, etc. Snapshot (minisequencing technology) is a multiplex analysis technology of SNPs developed by Applied Biosystems (ABI), USA, which can realize mid-throughput typing of SNPs. Its basic principle and process: firstly, multiplex PCR is conducted on a DNA template to generate target SNP amplified fragments; then the unbound primers and the remaining dNTPs are degraded by adding exonuclease I (Exo I) and shrimp alkaline phosphatase (SAP) to purify the PCR products so as to avoid interfere with a subsequent SBE reaction; then, the 3′ terminal of the primer directly binds to the target SNP, and is extended by TaqDNA polymerase, so that PCR reaction is carried out with this enzyme in combination with a ddNTP with a fluorescent label and the primer of which the 5′ terminal was close to a SNP locus; and finally, gene typing and data analysis are carried out by using a sequencer and software such as GeneScan. The SNaPshot has the characteristics of high sensitivity, good repeatability, and no requirement of additional equipment, and has been widely used in research fields such as forensic identification and SNP detection of human genes. It has been reported in the research field of plant genetics such as SNP typing and marker development, molecular marker-assisted breeding and genetic diversity analysis, showing the broad application prospects of the SNaPshot technology in plant genetics research. Up to now, the SNP marker is seldom applied in aspects of evaluation of genetic diversity of Pisum sativum L. and study of population genetic structure, with only sporadic reports of it, while the research of Pisum sativum L. in the genetic aspect by utilizing the SNP markers developed based on the SNaPshot technology is even less reported.

### SUMMARY

The present invention provides a set of neutral SNaPshot markers of Pisum sativum L. and use thereof in analysis of genetic diversity and population genetic structure. In the present invention, 432 accessions of Pisum sativum L. germplasms are used as test materials, and subjected to analysis of genetic diversity and population genetic structure of Pisum sativum L. through a set of neutral SNaPshot markers (46 neutral markers) of Pisum sativum L. The neutral markers can better group the Pisum sativum L. germplasms according to their geographic origins due to their independence of functional genes such as heat tolerance, and are more consistent with a type of sowing date.

A first objective of the present invention is to provide a set of neutral SNaPshot markers of Pisum sativum L., which consists of 46 neutral SNaPshot markers shown in Table 2, wherein peripheral amplification primer sequences and single base extension primer sequences of the 46 neutral SNaPshot markers are shown in Table 3.

A second objective of the present invention is to provide use of the aforementioned 46 neutral SNaPshot markers in analysis of genetic diversity and population genetic structure of a Pisum sativum L. population.

A third objective of the present invention is to provide a method for analyzing genetic diversity of Pisum sativum L. by employing the aforementioned 46 neutral SNaPshot markers, including:
1) SNaPshot PCR reaction
conducting peripheral amplification by using DNAs of the population of Pisum sativum L. germplasms to be tested as PCR templates with each locus being subjected to single amplification, purifying PCR products and then conducting SNaPshot PCR of them by employing single base extension primers, and detecting reaction products of the SNaPshot PCR by capillary electrophoresis via an ABI 3730XL DNA analyzer; and
2) data analysis
conducting data analysis of SNP loci by utilizing Gene mapper 4.1, wherein each sample is genotyped according to peaks corresponding to the SNP loci, and the resultant analysis results are a file of an Excel format and a peak map of a PDF format, and calculating genetic diversity parameters of two groups of SNP markers by utilizing PowerMarker 3.25, wherein the genetic diversity parameters includes a number of genotypes (NG), a major allele frequency (MAF), a number of alleles (NA), gene diversity (GD), expected heterozygosity (He) and polymorphic information content (PIC).

An amplification system for the aforementioned peripheral amplification in the step 1) is 35 μl in total: 30 μl of 1.1×T3 Super PCR Mix; 2 μl of 10 μM Primer F; 2 μl of 10 μM Primer R; and 1 μl of a Template (gDNA). Amplification procedure: 98° C. for 3 min; 98° C. for 10 s, 57° C. for 10 s, 72° C. for 15 s, 35 cycles; 72° C. for 2 min; storage at 4° C.

SNaPshot PCR is conducted with the single base extension primers in the aforementioned step 1). The PCR system is 5 μl in total: 2 μl of ABI SnapShot multiplex Mix (Applied Biosystems, Foster City, CA, USA); 1 μl of the Primers; 1 μl of a purified Post-PCR Template; and 1 μl of ddH₂O. Amplification procedure: 96° C. for 2 min; 96° C. for 10 s, 50° C. for 5 s, 60° C. for 30 s, 30 cycles; 60° C. for 30 s; and storage at 4° C.

The present invention further provides a method for analyzing a genetic structure of a Pisum sativum L. population by employing neutral SNaPshot markers of Pisum sativum L., comprising, on the basis of the aforementioned step 2), firstly conducting Bayesian cluster analysis by utilizing Structure 2.3.4, and determining an optimal population structure and population size according to a Delta K (ΔK) value; secondly, conducting principal coordinate analysis (PCoA) to check whether a result of the Structure analysis of Pisum sativum L. is reasonable; and finally constructing a phylogenetic tree by utilizing UPGMA cluster analysis to display the analysis result intuitively.

In the present invention, the Pisum sativum L. germplasms are divided into two genetic subpopulations A and B.

The technical effects of the present invention are as follows.
1. In the present invention, the SNaPshot method was introduced into the identification and evaluation of Pisum sativum L. germplasms for the first time, and a set of neutral SNaPshot markers (46 neutral markers) of Pisum sativum L. is developed to conduct analysis of genetic diversity and population genetic structure of Pisum sativum L. The neutral markers can better group the Pisum sativum L. germplasms according to their geographic origins due to their independence of functional genes such as heat tolerance, and are more consistent with a type of sowing date.
2. In the present invention, the genetic diversity evaluation and population genetic structure analysis of the 432 accessions of Pisum sativum L. germplasms are carried out by utilizing the neutral SNaPshot markers. After analysis of the neutral SNaPshot markers, it was found that the number of the markers significantly affected the total amount of NG and NA, and had a certain impact on the means of MAF, GD and PIC, but had little impact on the mean of He. When the number of markers were increased, the total amount of NG and NA was increased, but the mean of MAF was decreased, the means of GD and PIC were increased, and the proportion of markers with high and medium PIC was increased; and vice versa. From the inside of the marker, the population size has little impact on the total amount of NG and NA, indicating that the selection of the neutral markers was scientific and the distribution on the chromosome was uniform.

### BRIEF DESCRIPTION OF THE DRAWINGS

FIG. 1A shows the ΔK in the analysis of the neutral SNaPshot markers of Pisum sativum L. by Structure;

FIG. 1B shows the ΔK in the analysis of the neutral SNaPshot markers of Pisum sativum L. by Structure;

FIG. 1C shows the ΔK in the analysis of the neutral SNaPshot markers of Pisum sativum L. by Structure;

FIG. 1D shows the ΔK in the analysis of the neutral SNaPshot markers of Pisum sativum L. by Structure;

FIG. 2A shows analysis of population genetic structure of the neutral SNaPshot markers in 432 accessions of Pisum sativum L. germplasms, including analysis of 46 neutral SNaPshot markers by Structure;

FIG. 2B shows PCoA of 46 neutral SNaPshot markers;

FIG. 2C shows a genetic distance based on Nei and an UPGMA phylogenetic tree of 46 neutral SNaPshot markers;

FIG. 3A shows the population genetic composition of 432 accessions of Pisum sativum L. germplasms, wherein the population genetic composition is the genetic composition of Pisum sativum L. germplasms sown in spring (n=246) based on 46 neutral SNaPshot markers; and

FIG. 3B shows the population genetic composition of 432 accessions of Pisum sativum L. germplasms, wherein the population genetic composition is the genetic composition of Pisum sativum L. germplasms sown in winter (n=186) based on 46 neutral SNaPshot markers.

### DESCRIPTION OF THE EMBODIMENTS

### Example 1

### 1. Materials and Methods

### 1.1. Plant Materials

432 accessions of Pisum sativum L. germplasms from the National Crop Germplasm Bank of the Institute of Crop Science, Chinese Academy of Agricultural Sciences (Beijing, China) were selected as test materials, of which 363 accessions (84.0%) were from 22 provinces, cities and autonomous regions in China, 61 accessions (14.1%) were from 10 countries and organizations other than China, and the remaining 8 accessions (1.9%) were of unknown origin and were classified into the category of “unknown”. All Pisum sativum L. germplasms were divided into two categories according to the type of sowing date, wherein 246 accessions (56.9%) were of spring sowing type, and 186 accessions (43.1%) were of winter sowing type (Table 1).

TABLE 1
Sources and types of sowing date of 432 accessions
of Pisum sativum L. germplasms

Type of sowing date

| | Number | Spring | Winter |
|---|---|---|---|
| Source | (accession) | sowing | sowing |
| Qinghai, China | 63 | 63 | |
| Shaanxi, China | 48 | 1 | 47 |
| Inner Mongolia, China | 40 | 39 | 1 |
| Shanxi, China | 30 | 29 | 1 |
| Sichuan, China | 29 | 1 | 28 |
| Xinjiang, China | 23 | 23 | |
| Hubei, China | 19 | 1 | 18 |
| Gansu, China | 16 | 15 | 1 |
| Guizhou, China | 16 | | 16 |
| Anhui, China | 15 | 1 | 14 |
| Guangxi, China | 13 | | 13 |
| Henan, China | 13 | 2 | 11 |
| Chongqing, China | 12 | | 12 |
| Tibet, China | 7 | 7 | |
| Liaoning, China | 5 | 5 | |
| Hunan, China | 4 | 2 | 2 |
| Yunnan, China | 4 | 2 | 2 |
| Jiangsu, China | 2 | 2 | |
| Beijing, China | 1 | 1 | |
| Hebei, China | 1 | 1 | |
| Ningxia, China | 1 | 1 | |
| Shanghai, China | 1 | 1 | |
| Total number in China | 363 | 197 | 166 |
| United States | 18 | 11 | 7 |
| Germany | 11 | 11 | |
| United Kingdom | 7 | 1 | 6 |
| Bulgaria | 3 | 3 | |
| Canada | 3 | 3 | |
| France | 3 | 2 | 1 |
| India | 3 | 3 | |
| Turkey | 2 | 1 | 1 |
| Denmark | 2 | 2 | |
| Poland | 2 | 2 | |
| Nepal | 1 | 1 | |
| Netherlands | 1 | | 1 |
| IGARDA | 1 | 1 | |
| Syria | 1 | 1 | |
| Russian Federation | 1 | 1 | |
| Hungary | 1 | 1 | |
| Chile | 1 | 1 | |
| Total number in | 61 | 45 | 16 |
| foreign countries | | | |
| Unknown | 8 | 4 | 4 |
| Total number of | | 246 | |
| spring sowing | | | |
| Total number of | | | 186 |
| winter sowing | | | |
| Total number | 432 | | |

### 1.2 SNaPshot Analysis

Genome DNAs were derived from 432 accessions of Pisum sativum L. germplasms. The tender leaves of 3 plants were collected from each accession of material at 4 weeks after sowing, and mixed and extracted by a TSINGKE plant DNA extraction kit (Tsingke Biotechnology Co., Ltd., Beijing).

The design of peripheral primers followed the following principle: the primer length was 15-30 bp, and its effective length was generally no more than 38 bp. The GC content should be in 40%-60%, and the optimum Tm value should be in 58-60° C. The primer itself could not contain a self-complementary sequence. There should be no more than 4 complementary or homologous bases between the primers, and especially the complementary overlap at the 3′ terminal should be avoided.

The design principle of single base extension primer: the primer had a length of 15-30 bp, a GC content of 40%-60%, and an optimum Tm value of 58-60° C. PolyCs or PolyTs of different lengths were added to 5′ terminal of the primers, so that each primer could be distinguished by length. The shortest design of the tailed primer was 36 bp, and the lengths of the primers of two adjacent SNP loci generally differed by 4-6 nucleotides.

A GenoPea 13.2K SNP chip developed by Tayeh et al. was utilized, and the selected 46 loci were all of neutral mutations. For each SNP locus sequence, a pair of peripheral amplification primers and one single base extension primer were designed by utilizing Premier 5. See Tables 2 and 3 for SNP loci and SNaPshot primer information.

TABLE 2
SNP locus information

| | | | | | Predicted | | Predicted |
|---|---|---|---|---|---|---|---|
| Serial | Marker | | | Position | SNP | Predicted | protein |
| Number | Name | Sequence | LG | (cM) | position | SNP effect | function |
| 1 | PsCam036172_ | TTTTGCTTGTCCAATATTGTATTGTGAGTGCTG | 1 | 0.8 | of gene; | — | UDP- |
| | 21320_ | GTTTTGAACCTGAAATTTTTTGTGGTG[A/G]T | | | non-protein | | glucuronosyl- |
| | 1822 | CAACGATTCAGAAACATGAATTATTATTTTGT | | | coding | | transferase |
| | | CTTTAACTTGTATGTTTGTCTTAGGAT | | | sequence | | |
| 2 | PsCam035943_ | CTTTTCATCAAACGAGTTCATCGAATCCAAGC | 1 | 11 | Protein | Synonymous | Penta- |
| | 21097_ | TTGTGATTCTCTACGCAAAATGCGGCCT[T/C] | | | coding | substitution | tricopeptide |
| | 618 | GCGGATATCGCGGTTCATCTCTTTCGTAACGT | | | sequence | | repeat- |
| | | TGTTCAAAACCAGAATCTCTTCTCTTGG | | | | | containing |
| | | | | | | | protein |
| 3 | PsCam057738_ | CACCGAGATGGAACGTTGAAGCAAAAGGTT | 1 | 24.1 | of gene; | — | Cytosolic |
| | 38296_ | GCAGAGAATCCCGAGAGGTATAGATGTAAA[A/G] | | | non-protein | | purine 5′- |
| | 1700 | TCGCTAGTGTATGAGTTCGCGATGAAATT | | | coding | | nucleotidase |
| | | ATCTGACTTGCCGTTTTTCTTTGAAATTTAA | | | sequence | | |
| 4 | PsCam037986_ | TATATCTTAGAACCAATCATCAGCAATGTTGT | 1 | 40.5 | of gene; | — | HVA22- |
| | 23038_ | TTGAATATCCTTAACTTTAGATTTCTGC[T/C]G | | | non-protein | | like protein |
| | 1304 | TCGTTTGTCCTTATTTTGAATCTGTTGTTGCA | | | coding | | a |
| | | GGCTCCAATGGTGGCCTCATGTGAAAG | | | sequence | | |
| 5 | PsCam043062_ | CTTAGTTTCTAATTCATCCACATGATGATTTTT | 1 | 64.8 | of gene; | — | 60S |
| | 27092_ | GTTAGACAATTTTTGTTTGAGATTCTC[T/C]TT | | | non-protein | | ribosomal |
| | 1991 | ATTAAGAAAATGCTTAGTCTTAAACATGGATC | | | coding | | protein L13 |
| | | TTCATGGCTATTGTTATTTTGTGAAC | | | sequence | | |
| 6 | PsCam049876_ | AAGCACATTCAAAATTTATTTCTCAAACATCT | 1 | 85.4 | of gene; | — | |
| | 32490_ | CAAGGCAATTAGGATCAAATTCAGGCAC[T/G] | | | non-protein | | |
| | 1287 | GAAGTACCGAAACAAACCTGAACTGCAGCT | | | coding | | |
| | | CTATAGAAGAGTTCCTCTCCCACTGAACTT | | | sequence | | |
| 7 | PsCam051455_ | TGTACAATCATTATAAATATAGCTATTGTGAAG | 2 | 11.3 | of gene; | — | MTD1 |
| | 33948_ | AGATGTTATGCGGAAGAAAAGAAAAAC[T/G] | | | non-protein | | |
| | 107 | AAGCTATGATTTTATTCTACAACCTCTTTCTTG | | | coding | | |
| | | CTCAAACAAATAGAACTAGAGCAAGAT | | | sequence | | |
| 8 | PsCam045637_ | GGACAAGAAATCAGTTTTGATGATTTATCTCT | 2 | 26.8 | Protein | Nonsynonymous | Kinase-like |
| | 29281_ | TGAAGAGAAGAAACAATTTCAAAGAGCT[A/G] | | | coding | substitution | protein |
| | 840 | TTGCTTGTGGGGAATTAAGCAAGATGATCA | | | sequence | | |
| | | CACCATGGGATCCATGGTGGTCAAAGCATT | | | | | |
| 9 | PsCam025478_ | AGTTAGACTTTGGCTAATTGGCATTGCTTCTG | 2 | 43.6 | Protein | Synonymous | |
| | 14590_ | TCTTTTTTCTGTTTCAGTTTCCAATTGC[A/G]C | | | coding | substitution | |
| | 1802 | TTTTGGTATTCGGCTTTGAAGCAGTAAAGTTG | | | sequence | | |
| | | TACAAGGATCACAGAAAGAGGAAGGGC | | | | | |
| 10 | PsCam041441_ | ACGTCAAGGCAGCGAGAGATATGGTGTCATC | 2 | 51.8 | of gene; | — | cyanate |
| | 25895_ | CGAAAGCCGAAACTCTACGAGAGGTGAAA | | | non-protein | | hydratase |
| | 460 | [T/C]TTCGTTGAAGTAAACTTCTACCTTAATCAA | | | coding | | |
| | | AAATTGAGGAAGAAATATTTTTTGAGATGT | | | sequence | | |
| 11 | PsCam050369_ | AACTTTCAGGGCGAGGTGATTCATAACTGGA | 2 | 65.5 | of gene; | — | |
| | 32956_ | ACTTTCAGAGGTAGGTGATTCATACAAAC[T/C] | | | non-protein | | |
| | 339 | GGCACTTTCAGAGCTTTCAAAATCAGGAACT | | | coding | | |
| | | TCGCCATCAAAGGAAGTATCAGGAGCATC | | | sequence | | |
| 12 | PsCam040267_ | CATCATGTGCTCAACTTTGGTCACATTTTCCA | 2 | 84 | of gene; | — | |
| | 25050_ | CGTTTCTCAGGTGCTAAGAAGTTCAAAA[T/C] | | | non-protein | | |
| | 210 | CAAATAGTCTTCCCTCCATCTCCTCTTCCGCG | | | coding | | |
| | | CCGAAGGTCGCCTCTGGCGGCGGCGGAC | | | sequence | | |
| 13 | PsCam000362_ | CGTATTTTGGTAACACACCACCAAATCAGAG | 2 | 101.8 | Protein | Synonymous | Defects in |
| | 321_ | AGTCTCGATGCTGAGATTCCGGAATCAAC[A/G] | | | coding | substitution | morpholog |
| | 595 | CACGCTGTTTGTAGATCTTTGCATAAGAAA | | | sequence | | protein- |
| | | CGTCGAGGATTTAACTCGAAATAATCGAAT | | | | | like protein |
| 14 | PsCam034276_ | AAATCCACATTAGGTCTCTCTACTTTCATGTC | 3 | 7 | Protein | Nonsynonymous | flowering |
| | 19685_ | CATATCCAAATCAGAACCCCTCCCCTCA[T/C] | | | coding | substitution | locus d |
| | 1661 | ACACATTGTCCTTATTCAACCAAGATCTTTCA | | | sequence | | |
| | | TCCAAACCTTCATACTGGCAAGAAACAT | | | | | |
| 15 | PsCam000088_ | GAGGAATTATGGAGAGACAATGAGACATGGA | 3 | 38.3 | of gene; | — | PRLI- |
| | 75_ | GAAAGACAGCTACAAAACAAAAAACAAAA | | | non-protein | | interacting |
| | 1456 | [A/G]TGCAATTGAACTTCACTATGATACTGTTT | | | coding | | factor G |
| | | TTGTTTCTTACATGGTTGTCTGTTTTTGTGT | | | sequence | | |
| 16 | PsCam037526_ | TTCTTCTTCTTCAAGCTGCTTAAGCCAAAGA | 3 | 43 | Protein | Synonymous | Mucin- |
| | 22605_ | ATGGCATGAACCCTCTGTCTCATAATCCT[A/G] | | | coding | substitution | like protein |
| | 590 | TAATCCTTAGCAAGCTTCTCAACAGTATAAAC | | | sequence | | |
| | | CTCAGGATCCTTTTTGTGCAAGCGATAC | | | | | |
| 17 | PsCam055871_ | GCTTCCAGAATAGCTTGATTAGCGGTGAAGT | 3 | 65.4 | of gene; | — | putative |
| | 36784_ | GAGCGAATTTGAGATAAGGAGAAGACTCG[T/G] | | | non-protein | | gibberellin |
| | 1179 | AAAAGTGCATGTGAAGTGCATCAGACAAA | | | coding | | signaling |
| | | GATGAATCCAGAGTTTCATCAGGGGAAACTC | | | sequence | | DELLA |
| | | | | | | | protein LA |
| 18 | PsCam043018_ | AGCCACTAATTGACAATTAGACATAGTTGTAT | 3 | 101.2 | of gene; | — | Proliferation- |
| | 27052_ | ATGTATATATGTTAGTGAGGACAATAAG[A/G]A | | | non-protein | | associated |
| | 1394 | AAGAAAATCAGATTGTATATACATTAAGCGTG | | | coding | | protein |
| | | GTGAAGAAATATATGTGAAAAAGCCTT | | | sequence | | |
| 19 | PsCam040468_ | CTAATTTAATCAACTTTCATTCAAAAGCAAAA | 3 | 133.5 | of gene; | — | putative |
| | 25210_ | CACCTTGCAGCATAGCACAACTGCAAGA[A/C] | | | non-protein | | His-Asp |
| | 90 | GAGCACAATGGCACTATATACATAAGAGAGG | | | coding | | phosphotransfer |
| | | TTTTAATCACCCTAAACAACCAAAAAAGA | | | sequence | | protein |
| 20 | PsCam0 | AGAATGCAATCTGACGAAAGCCATGAGGCAC | 4 | 2.7 | of gene; | — | putative |
| | 36980 2 | GAAGTTTATACCATCATCCACTAAAAACA[T/C] | | | non-protein | | adenosine |
| | 2091 68 | GCCACGTAATCATGCCAAAACTCCGTCCAGG | | | coding | | 5′- |
| | | TTCAATAAACCGCCACAATAGAGAAACCT | | | sequence | | phosphosulphate |
| | | | | | | | reductase |
| 21 | PsCam004622_ | CATGGATCCTCTGGAGTCTTTCTTCGCGTCCT | 4 | 17.8 | Protein | Synonymous | Protein |
| | 3470_ | GACTTTTTAACGAGTGAAATAGTTGGAC[A/G] | | | coding | substitution | CHUP1 |
| | 1686 | GTTGCAGAAGCCTTATGAGTGTTGGCCAATTT | | | sequence | | |
| | | TGCCAAAGGCCGAGACGGAATCGGCGGT | | | | | |
| 22 | PsCam035376_ | CTGATACACCTTACCCTCCGCAAAAGTCAAA | 4 | 32.8 | Protein | Nonsynonymous | G patch |
| | 20564_ | CAAAGATGCTTCCCAGAAAGAAAATGTTA[A/G] | | | coding | substitution | domain- |
| | 2643 | TGAATATGCAAAACATGACATCATAAGTGG | | | sequence | | containing |
| | | TGAAAGCAACAGTGTGGTGTCACTGAAACA | | | | | protein |
| 23 | PsCam044939_ | CATGTCATAGGGGCCTAGACCGTAACCCATCA | 4 | 44.4 | Protein | Synonymous | Retinoblastoma- |
| | 28681_ | TATGCATTGGACCAGCAGCAGCATATGG[T/G] | | | coding | substitution | binding |
| | 1017 | GCCATAAATCCATCCATACCAGGTTGGATACC | | | sequence | | protein |
| | | ATTCCAGTATGGGTTATAACCAGGAGGT | | | | | |
| 24 | PsCam005290_ | ACAGAAAGTCAAGATCGCAGAAAAGATATTC | 4 | 61.4 | Protein | Synonymous | RING-H2 |
| | 4012_ | CCGATGATAACTGAGACGACGAGGACACC[A/ | | | coding | substitution | finger |
| | 1343 | GJACCCATTTGACCCTCTCAACGAAATTATCA | | | sequence | | protein |
| | | AAAAATTCCAGTAAAGAGAATTGATTTGCC | | | | | ATL4M |
| 25 | PsCam026873_ | TTGAGAACACTTGCAATTTCAACAATCACAT | 4 | 78.15 | Protein | Nonsynonymous | Annexin |
| | 15607_ | GATAATCTCTGGAAACATTCTTAAGAGCT[A/G] | | | coding | substitution | |
| | 1235 | CATTTGCCAACACAGCATAACAATCTGAAGG | | | sequence | | |
| | | TTCCAATATCCACCTATACATTGCTTTCT | | | | | |
| 26 | PsCam042409_ | TTTAATCTTATATATACATACATGAAGGAAAAA | 4 | 90.5 | of gene; | — | Calcium- |
| | 26473_ | AAATATAGAATCAAAAAATTGTACCAT[T/C]A | | | non-protein | | transporting |
| | 1566 | GAAGAAGCAGATAAGGTTGGAATTTGTGAGC | | | coding | | ATPase |
| | | TTGGAAACCAAGCAAAGAATTATCCTAA | | | sequence | | |
| 27 | PsCam043345_ | CACAGCGCAACGACCGTGGGACCCACCTCTC | 4 | 111.9 | Protein | Synonymous | |
| | 27360_ | GTTCAAGAGGGTCGGGTTCGGTCCTTCTG[T/C] | | | coding | substitution | |
| | 370 | ACGGGTCGGAAGTTTCGCAATCTTTTCAAA | | | sequence | | |
| | | TATCCGCTTCATCGTGTCTTCGTTTCACCG | | | | | |
| 28 | PsCam006884_ | GGAATCATGACAGGTTCGATGGAACCGTATTT | 5 | 7.4 | Protein | Nonsynonymous | GH3 |
| | 5125_ | GGAAAAACTGAGACACTATGCAGGTGTG[A/G] | | | coding | substitution | family |
| | 2126 | TACCTTTGTTGACCGCTGATTACGGAGCCTC | | | sequence | | protein |
| | | TGAAGGATGGATAGCTGCAAATGTGAATC | | | | | |
| 29 | PsCam057416_ | AAAAAAATAACAGACTCAAACTCTATCCAAT | 5 | 26.8 | of gene; | — | |
| | 38023_ | GGGATATTAAAGCAATACGCAACCAAAGA[T/C] | | | non-protein | | |
| | 271 | GGAAGCAGGCACAAACGACCAGCATTTTT | | | coding | | |
| | | GGAAAGTGCACTCTTGTGGCAAATGATTGCG | | | sequence | | |
| 30 | PsCam004972_ | ATTTCCTTATTTATGTGTCAAAAGATCGATCGC | 5 | 42.9 | of gene; | — | Mitochondrial |
| | 3765_ | GCAAAATAAAATAAAATACAAATCTCC[A/G]C | | | non-protein | | inner |
| | 1941 | CGATACAATGTATTTTCTTTTCCACAAACAAA | | | coding | | membrane |
| | | ACAAAACAAAAGTTACCATTTTCTTCT | | | sequence | | magnesium |
| | | | | | | | transporter |
| | | | | | | | mrs2 |
| 31 | PsCam049238_ | AATCATCAACAAAAATACATAGCGAGACTTCT | 5 | 60.3 | of gene; | — | Nuclear |
| | 31877_ | CTATACTCTTTTATGATTTTCGAATATG[A/G]AC | | | non-protein | | cap- |
| | 3224 | CATAACTACAAAATAAACCACAAGTGGCAAT | | | coding | | binding |
| | | GCAGTAATGCATAAATACCTGTTTGAC | | | sequence | | protein |
| | | | | | | | subunit |
| 32 | PsCam049156_ | TTTTGGAGCCAAGGATGCTCTGCAAGACAAG | 5 | 77 | of gene; | — | Calcium |
| | 31799_ | TAAAGGAAAACATCATGAAGTCAGTGAAT[T/C] | | | non-protein | | and |
| | 1877 | TATTCTGATAAATCCTTAACATAAACAGCAT | | | coding | | calcium/cal |
| | | AAATGATTTCTAATCATTATTTATTTCAG | | | sequence | | modulin- |
| | | | | | | | dependent |
| | | | | | | | serine/thre |
| | | | | | | | onine- |
| | | | | | | | protein |
| | | | | | | | kinase |
| 33 | PsCam054451_ | AGCATGGCCATCCCCAAAAAGGGAACGAGA | 5 | 108.7 | of gene; | — | |
| | 35939_ | GCTGAGAGCAGAACGGCCGATAATTAGTGA | | | non-protein | | |
| | 483 | [T/C]GCGGACACTAAAACATTATCAAAATTCAT | | | coding | | |
| | | GAAGTGTTCATATTAAGAAATAAAAATCATA | | | sequence | | |
| 34 | PsCam034709_ | GTGTTTGAAGTTCCGATTGAAAAGTTGGAAA | 6 | 2.3 | Protein | Nonsynonymous | Elongation |
| | 20077_ | TCGAATTCAGAAAGCCTGTAGATCAGTTA[A/G] | | | coding | substitution | factor 1- |
| | 714 | AGAAGGAAACAATATCGGAGTCAGAGAAG | | | sequence | | alpha |
| | | CCTTTTGTGAACGAACTGACAATGAAAAACG | | | | | |
| 35 | PsCam055500_ | TTTGTTTTCCACGAAAGAACTAAACACATTA | 6 | 13.2 | of gene; | — | Stomatin- |
| | 36558_ | AAGTGGATTGTCATTTCATAAGAGACCAT[A/G] | | | non-protein | | like protein |
| | 381 | TTTAATCAGGTGTCATCACTATGACTCATGTT | | | coding | | |
| | | TCAACTCATCAGCAATAGGCTGACATCT | | | sequence | | |
| 36 | PsCam033926_ | GGTTTATAGTCGTCCAAAAACTAAGGTAGCG | 6 | 32.7 | Protein | Synonymous | golgin |
| | 19423_ | GTAGTACCGTCAGAAATAGAGCAGAAGAG[T/C] | | | coding | substitution | candidate 6 |
| | 1600 | GGTGAAAGTGATGGTGAATATATTAAGCGG | | | sequence | | |
| | | CTGAAAGCTTTTGTTGAGAATCAGCACTCT | | | | | |
| 37 | PsCam051338_ | ACAAGCACAGGAAAGGTTACTAATCATACCT | 6 | 40.2 | Protein | — | Reticulon- |
| | 33847_ | ATTTCTAAGTGTGGCATAGTTAGAATGCA[A/G] | | | coding | | like protein |
| | 320 | AAAGAGTAGTACGATCAAAAGCAGCAAGAC | | | sequence | | B16 |
| | | ATCAGAACAAATTGTTAAGAATGGTAAATC | | | | | |
| 38 | PsCam037094_ | GGAACATTTTGTCTGTCTAGGACCTTTGTAGC | 6 | 63.2 | of gene; | — | DNA |
| | 22201_ | ACCGACACGTCTGAATTTTTTTTTGGAA[T/C] | | | non-protein | | replication |
| | 1066 | GATTGGATTGAATGACTTAATCAGAGTATGAG | | | coding | | protein- |
| | | GATCATTCCTGTAGTACCTTTTTTCAAC | | | sequence | | related |
| 39 | PsCam004372_ | TGAAGGAGTTGGACAATTATTCGTTGGTGTC | 6 | 90.3 | Protein | Synonymous | serine- |
| | 3289_ | GAATACGAGGGAATGACTTGAGGAGCTTT[A/G] | | | coding | substitution | threonine |
| | 2443 | CAGTAAACCTCATTAGAAACTCCATTTTCA | | | sequence | | protein |
| | | AGACAAATCTTGTTTTGACCCAATCTGCAA | | | | | kinase |
| 40 | PsCam014062_ | ATGTAAAGACTATGGAAGAGCTTACCTGATC | 6 | 97.1 | Protein | Nonsynonymous | Metacaspase- |
| | 9583_ | AATTTTACTTCGAAAAATACGTTTCAAGA[T/G] | | | coding | substitution | 1 |
| | 426 | CCTGTTAAAGTGTTTGTTTTGGTGAATCTTCC | | | sequence | | |
| | | CAATTTCGTCGTGCAGTTTTTCTAGCAA | | | | | |
| 41 | PsCam010804_ | AACTCCTTCCTAAGTTCTGAGAAAGTTTAAG | 7 | 7.3 | of gene; | — | Carbon |
| | 7280_ | TTATGAAAAATATAATGTTTAAGGCCTTA[A/G] | | | non-protein | | catabolite |
| | 996 | TGATGACAATTACCCAATAATAAGATAAGTTG | | | coding | | repressor |
| | | AATGCATATGATGCAGGTACTCTGACGG | | | sequence | | protein- |
| | | | | | | | like protein |
| 42 | PsCam040802_ | ATCTGTAAATTCAGCAACACACATTGTTGGA | 7 | 15.9 | Protein | Synonymous | blue Cu |
| | 25456_ | GGCAAGGTTGGTTGGAATTTGCCTAGTTA[T/C] | | | coding | substitution | protein |
| | 306 | TATAGTTTCTTTGAGGATTGGTCAAAGAACC | | | sequence | | |
| | | AAACCTTCATTGTTGGCGATCAACTTCGT | | | | | |
| 43 | PsCam001376_ | TGTTGTCACTATCAAAGACAAATTTGATGATA | 7 | 34 | of gene; | — | Cytochrome |
| | 1148_ | GTGAGATAGGAAAGGAATAAAACACAGT[A/G] | | | non-protein | | P450 |
| | 333 | TTTAGATCATAGATAATATTTGTACACTTAAA | | | coding | | |
| | | GTTTTAACATTTGTGACACCTAGTTCAA | | | sequence | | |
| 44 | PsCam000349_ | CAAATGACCCATTCAATCATTTTCTGTTGGCC | 7 | 46 | of gene; | — | Reticuline |
| | 309_ | ATGTTGCCTAAGCCTTGTTTGGCAGTGT[T/C] | | | non-protein | | oxidase |
| | 200 | GGGATACTTTGTTCATTCCAGAAAAAGTTTTG | | | coding | | |
| | | AGGATCAACTTGAGTCTTAACCTTATTT | | | sequence | | |
| 45 | PsCam037467_ | TCTAAAATACATTCCTGATGGAAACTACATAA | 7 | 58.1 | Protein | Synonymous | Receptor- |
| | 22549_ | AGGTTGGAAGCGTCGCCACAATCAACAA[A/G | | | coding | substitution | like protein |
| | 557 | JCCAGACTTGTTGCCAACACTCTCCACATTGC | | | sequence | | kinase |
| | | GTTACTTTACCAACACGTTATCTAAAAAA | | | | | |
| 46 | PsCam024028_ | CCTGTTCTTCACATTGAATCCCTTGGTCATGC | 7 | 95.7 | of gene; | — | Beta- |
| | 13660_ | CCTTCATGCTTTTATAAACGGGAAACTC[A/G] | | | non-protein | | galactosidase |
| | 713 | CAGGTATCTTTCTTTACTTCAATGAAGAATCT | | | coding | | |
| | | CATGAATCATGTAATGATATATCGATAT | | | sequence | | |

TABLE 3
SNAPshot primer information

| | | Names of | | Names of | |
|---|---|---|---|---|---|
| | | peripheral | | SNP single | |
| | | ampli- | | base | Sequences of SNP |
| Serial | Marker | fication | Sequences of peripheral | extension | single base |
| Number | Name | primers | amplification primers | primers | extension primers |
| 1 | PsCam036172_ | 1-F | TCCTAAACACAGCACTCAACAC | 1-SNP-F | TTTTTTTTTTACCTGAAATTTT |
| | 21320_ | 1-R | ACCCCACCAGATTGAGATGA | | TTGTGGTG |
| | 1822 | | | | |
| 2 | PsCam035943_ | 2-F | CCCCAATCCCCTCCAAAACA | 2-SNP-F | TTTTTTTTTTTTTTTTTTTACG |
| | 21097_ | 2-R | GAGAGTCCCATTCGGGCTTG | | CAAAATGCGGCCT |
| | 618 | | | | |
| 3 | PsCam057738_ | 3-F | GCCGAAGCCTACTTGTTTGC | 3-SNP-F | TTTTTTTTTTTTTTTTTTTCCC |
| | 38296_ | 3-R | GCATTTCAAGCATGGGGACT | | GAGAGGTATAGATGTAAA |
| | 1700 | | | | |
| 4 | PsCam037986_ | 4-F | TGAGGTTGACGACTGCCTTT | 4-SNP-F | TTTTTTTTTTTTTTTTTTTTTT |
| | 23038_ | 4-R | GCGCAGCACCAAAATAAGGTA | | TTTTTTTATATCCTTAACTTTA |
| | 1304 | | | | GATTTCTGC |
| 5 | PsCam043062_ | 5-F | CCGAGGCAGAGAAGGAAGAC | 5-SNP-R | TTTTTTTTTTTTTTTTTTTTTT |
| | 27092_ | 5-R | TGTGCCCTTCATAACCACTGAT | | TTTTTTTTTTTTAAGACTAAG |
| | 1991 | | | | CATTTTCTTAATAA |
| 6 | PsCam049876_ | 6-F | ACGCCATTCCTCGAACATCT | 6-SNP-F | TTTTTTTTTTTTTTTTTTTTTT |
| | 32490_ | 6-R | GGCATGTCTCCTTTGCAGGT | | TTTTTTTTTTTTTTTTTTATTA |
| | 1287 | | | | GGATCAAATTCAGGCAC |
| 7 | PsCam051455_ | 7-F | GGTTCTGCATTTCCAAATCAACT | 7-SNP-R | TTTTTTTTTTTTTTTTTTTTTT |
| | 33948_ | 7-R | TCACTTCCACCTCTTTATCCGC | | TTTTTTTTTTTTTTTTTTAGGT |
| | 107 | | | | TGTAGAATAAAATCATAGCTT |
| 8 | PsCam045637_ | 8-F | TGGTTGGGTGAGGCTGATTT | 8-SNP-F | TTTTTTTTTTTTTTTTTTTTTT |
| | 29281_ | 8-R | CCGTTGTAGAGGCGAAGAGT | | TTTTAGAAGAAACAATTTCA |
| | 840 | | | | AAGAGCT |
| 9 | PsCam025478_ | 9-F | TGGTATTGGGTGCTCTTCGG | 9-SNP-F | TTTTTTTTTTCTGTTTCAGTTT |
| | 14590_ | 9-R | ATAAATCCGCCTCCCGAACC | | CCAATTGC |
| | 1802 | | | | |
| 10 | PsCam041441_ | 10-F | GGTTGTATTTCACTCTCCCGTT | 10-SNP-F | TTTTTTTTTTTTTTTTCGAAA |
| | 25895_ | 10-R | ATAGAGGGGTTGTGGGTCAAG | | CTCTACGAGAGGTGAAA |
| | 460 | | | | |
| 11 | PsCam050369_ | 11-F | TTGGATTCTTTACTGGGAGCTG | 11-SNP-F | TTTTTTTTTTTTTTTTTTTTTC |
| | 32956_ | 11-R | TGGGTTGGTGGCAGAATAGG | | AGAGGTAGGTGATTCATACA |
| | 339 | | | | AAC |
| 12 | PsCam040267_ | 12-F | GAAACAGGGAGGTGTGGGAG | 12-SNP-F | TTTTTTTTTTTTTTTTTTTTTT |
| | 25050_ | 12-R | TGGATTGACGTGGTTGTTTGG | | TTTTTTTTTTCAGGTGCTAAG |
| | 210 | | | | AAGTTCAAAA |
| 13 | PsCam000362_ | 13-F | TTATCGCTAGCAGGGGACAAC | 13-SNP-F | TTTTTTTTTTTTTTTTTTTTTC |
| | 321_595 | 13-R | GATACCGAGATACAGTTCCTGC | | TGAGATTCCGGAATCAAC |
| 14 | PsCam034276_ | 14-F | TCCTCAATTCCCTTCAGTGCC | 14-SNP-F | TTTTTTTTTTTTTTTTTTTTTT |
| | 19685_ | 14-R | TGGTAATGGAGGAGGCGAGA | | TTTTTTTTTTTTTTTTTTTTTT |
| | 1661 | | | | CAGAACCCCTCCCCTCA |
| 15 | PsCam000088_ | 15-F | GTCTGAAACCGACACCGACA | 15-SNP-R | TTTTTTTTTTTTTTTTTTTTTT |
| | 75_1456 | 15-R | CTGTCCAAGGAACAGCCTCA | | TTTTTTTTTTTTTTTTTTAGTA |
| | | | | | TCATAGTGAAGTTCAATTGCA |
| 16 | PsCam037526_ | 16-F | ACTCAGGGAAAGTGTCGAGC | 16-SNP-F | TTTTTTTTTTTTTTTTTTTTTT |
| | 22605_ | 16-R | TGGAAGAAACGAATGGCGGA | | TTTTTTTTTTTTTGAACCCTC |
| | 590 | | | | TGTCTCATAATCCT |
| 17 | PsCam055871_ | 17-F | GCCACTGCATCCCTTGTTTG | 17-SNP-F | TTTTTTTTTTTGAGATAAGGA |
| | 36784_ | 17-R | TCACAGGAAACTGGGGTTCG | | GAAGACTCG |
| | 1179 | | | | |
| 18 | PsCam043018_ | 18-F | ATGGGATCAGCTTCGGCTTC | 18-SNP-F | TTTTTTTTTTTTTTTTGTATAT |
| | 27052_ | 18-R | ACAAAGAAGAAGGGCGGTGG | | ATGTTAGTGAGGACAATAAG |
| | 1394 | | | | |
| 19 | PsCam040468_ | 19-F | TGAACCAGTAACACAAAACTACC | 19-SNP-F | TTTTTTTTTTTTTTTAGCATAG |
| | 25210_ | | A | | CACAACTGCAAGA |
| | 90 | 19-R | CTTTGGCAGCAGGAGTGAGA | | |
| 20 | PsCam036980_ | 20-F | GCCTGACGGAAGAACGGAA | 20-SNP-F | TTTTTTTTTTTTTTTTTTTTTT |
| | 22091_ | 20-R | AAGAAACAGCGAGAGCCATGA | | TTTTTATACCATCATCCACTA |
| | 68 | | | | AAAACA |
| 21 | PsCam004622_ | 21-F | GCTAACAGATGAGCCGAACG | 21-SNP-F | TTTTTTTTTTTTTTTTTTTTTT |
| | 3470_1686 | 21-R | ACCAATCAATGCCTCCTGCC | | TTTTTTTTTTTTTTTTTTTAAC |
| | | | | | GAGTGAAATAGTTGGAC |
| 22 | PsCam035376_ | 22-F | GAGCCGTTTGATTGCTGGTG | 22-SNP-R | TTTTTTTTTTTTTTTTTTTTTT |
| | 20564_ | 22-R | ACTGATCCTGTGGCAAACCA | | TTTTTTTTTTTTTGATGTCATG |
| | 2643 | | | | TTTTGCATATTCA |
| 23 | PsCam044939_ | 23-F | GAGGCGGAGGAGGAATAGGA | 23-SNP-F | TTTTTTTTTTTTTTTTTTTTTT |
| | 28681_ | 23-R | TTTCAGTGGAAACCCCCACA | | ACCAGCAGCAGCATATGG |
| | 1017 | | | | |
| 24 | PsCam005290_ | 24-F | CCAACACAACACATTAGAAGCTC | 24-SNP-F | TTTTTTTTTTTTTTTTTTTTTT |
| | 4012_1343 | | A | | TTTTTTTTTTTTTTTTTTTTTT |
| | | 24-R | TGCTGGTTGGTTGTTTGGTAG | | TTCTGAGACGACGAGGACAC |
| | | | | | C |
| 25 | PsCam026873_ | 25-F | CACATTCACAAGCTCTTCAGGT | 25-SNP-F | TTTTTTTCTCTGGAAACATTC |
| | 15607_ | 25-R | GGTGGGGAACCGATGAGAG | | TTAAGAGCT |
| | 1235 | | | | |
| 26 | PsCam042409_ | 26-F | CAACAACCACTTCCACAGATACA | 26-SNP-R | TTTTTTTTTTTTTTTTTTTTTT |
| | 26473_ | 26-R | TTCCATCCAACTCACACATCTC | | TTTTTTTTTTTTTTTTTTCCAA |
| | 1566 | | | | CCTTATCTGCTTCTTCT |
| 27 | PsCam043345_ | 27-F | ACTTCGATCCGCTATGGACAC | 27-SNP-F | TTTTTTTTTTTTTTTTTTCGGG |
| | 27360_ | 27-R | GTGCTTCGGGGCTATCATCG | | TTCGGTCCTTCTG |
| | 370 | | | | |
| 28 | PsCam006884_ | 28-F | TCTTACCAGCAGAGTCACGG | 28-SNP-F | TTTTTTTTTTTTTTTTTTACTG |
| | 5125_2126 | 28-R | TCTCAAGCTGCGAAAGAGGAA | | AGACACTATGCAGGTGTG |
| 29 | PsCam057416_ | 29-F | AGCCTCTAGGTATCCAGGCA | 29-SNP-F | TTTTTTTTTTTTTTTTTTTTTT |
| | 38023_ | 29-R | AGAGCTTATCGGCTTACAGCAT | | TTTTAGCAATACGCAACCAA |
| | 271 | | | | AGA |
| 30 | PsCam004972_ | 30-F | AACGCCCGAAACTCAAATGG | 30-SNP-F | TTTTTTTTTTTTTTTTTTTTTT |
| | 3765_1941 | 30-R | AGGTTGCCAGTGAAAGGAGA | | TTTTCAAAATAAAATAAAATA |
| | | | | | CAAATCTCC |
| 31 | PsCam049238_ | 31-F | AGACAGCAGGTGTTCGTTGT | 31-SNP-F | TTTTTTTTTTTTTTTTTTTTTT |
| | 31877_ | 31-R | GCCCCTCAACGTGTCTTTGT | | TTTTTTTTTATACTCTTTTATG |
| | 3224 | | | | ATTTTCGAATATG |
| 32 | PsCam049156_ | 32-F | AAGCCTTGACTTGACGACATCT | 32-SNP-F | TTTTTTTTTTTTTTTTTTTTTT |
| | 31799_ | 32-R | TGAATGGTTGAAGGAGAAGGGT | | TTTTTTTGAAAACATCATGAA |
| | 1877 | | | | GTCAGTGAAT |
| 33 | PsCam054451_ | 33-F | ATATCAATCTCGGATAGCAGCAC | 33-SNP-F | TTTTTTTTTTTTTTTTTTTTGA |
| | 35939_ | 33-R | CCGTTCCTTCACAGATGGGT | | ACGGCCGATAATTAGTGA |
| | 483 | | | | |
| 34 | PsCam034709_ | 34-F | CCAGGCACAGCAAGAGTTGA | 34-SNP-F | TTTTTTTTTTTTTCAGAAAGC |
| | 20077_ | 34-R | CAAACTCGATTTCAACGACGC | | CTGTAGATCAGTTA |
| | 714 | | | | |
| 35 | PsCam0555 | 35-F | TCCATGCACATTTCCTACACCT | 35-SNP-F | TTTTTTTTTTTTTTTTTTTTTG |
| | 00_36558_ | 35-R | CCCCCTTAAGTTGGAGAGTGA | | ATTGTCATTTCATAAGAGACC |
| | 381 | | | | AT |
| 36 | PsCam033926_ | 36-F | CTGTCAAAAGGCTGGAGGCA | 36-SNP-F | TTTTTTTTTTTTTTTTTTTTTT |
| | 19423_ | 36-R | ACAAAAGCGACAACCAAAAACGA | | TTTTTCGTCAGAAATAGAGC |
| | 1600 | | | | AGAAGAG |
| 37 | PsCam051338_ | 37-F | TGTTGGTGGTTGTCTGCTCA | 37-SNP-F | TTTTTTTTTTTTTTTTTTTTTT |
| | 33847_ | 37-R | GTTTCGTTCGCTGCCATTGT | | TTTTTTTTTAGTGTGGCATAG |
| | 320 | | | | TTAGAATGCA |
| 38 | PsCam037094_ | 38-F | AGCGAAGAGGATGACATGAGTA | 38-SNP-F | TTTTTTTTTTTTTTTTTTTTTT |
| | 22201_ | 38-R | TGCTTCGTCTGTTTCGGGAG | | TTTTTTTTTTTTTTCGTCTGA |
| | 1066 | | | | ATTTTTTTTTGGAA |
| 39 | PsCam004372_ | 39-F | GCGCATTTACAGTTTGGGCT | 39-SNP-F | TTTTTTTTTTTTTTTTTTTTTT |
| | 3289_2443 | 39-R | CGACCTCGAGATGGGAAACC | | TTTTTTTTTTTTTTTTTTTGGA |
| | | | | | ATGACTTGAGGAGCTTT |
| 40 | PsCam014062_ | 40-F | TGCGACGTAATTGCTCAAAGT | 40-SNP-F | TTTTTTTTTTTTTTTTTTTTTT |
| | 9583_426 | 40-R | AGGCTTTCGGAGGAAAACAGA | | TTTTTTTTTTTCTTCGAAAAA |
| | | | | | TACGTTTCAAGA |
| 41 | PsCam010804_ | 41-F | GGTGAACCCTTGGCAACTTC | 41-SNP-R | TTTTTCTTATTATTGGGTAATT |
| | 7280_996 | 41-R | ATGGTCGCTTCCCACTTTCT | | GTCATCA |
| 42 | PsCam040802_ | 42-F | TGGCTGAGAAAGTGAACCTTAGT | 42-SNP-F | TTTTTTTTTTTTTTTTTTTGGT |
| | 25456_ | 42-R | TGGTGTGTGTCGGTGGAAA | | TGGAATTTGCCTAGTTA |
| | 306 | | | | |
| 43 | PsCam001376_ | 43-F | ATAGACAACTAGAGATTGGTTTTT | 43-SNP-F | TTTTTTTTTTTTTTTTGATAGG |
| | 1148_333 | | GAAG | | AAAGGAATAAAACACAGT |
| | | 43-R | GGTTAACAATGTCAATGTACACAA | | |
| | | | TCA | | |
| 44 | PsCam000349_ | 44-F | AGGGCCAGAAGAAGTAACAAAA | 44-SNP-F | TTTTTTTTTTTTTTTTTTTTTT |
| | 309_200 | | G | | TTTTAAGCCTTGTTTGGCAGT |
| | | 44-R | TTGGGAAGGATCAGAAGCTGG | | GT |
| 45 | PsCam037467_ | 45-F | CTGTGGAGGCACAAATGAGGT | 45-SNP-R | TTTTTTTTTTTTTTTTTTTTTT |
| | 22549_ | 45-R | CACGCTCAACCTCTTCCCAT | | TTTTTTTTTTCGTCGCCACAA |
| | 557 | | | | TCAACAA |
| 46 | PsCam024028_ | 46-F | ACACGACGGCAGATAAAAGTG | 46-SNP-F | TTTTTTTTTTTTTTTTTTTTTT |
| | 13660_ | 46-F | GCGTTTCCGCTGTTTCCTAC | | TTTTTTTTTTTTTTTTTGCTTT |
| | 713 | | | | TATAAACGGGAAACTC |

The extracted DNA sample was diluted to 20 ng/μl and then used as a PCR template to conduct peripheral amplification with 1.1×T3 Super PCR Mix (Tsingke Biotechnology Co., Ltd., Beijing), wherein each locus was subjected to single amplification, and each pair of primers was amplified according to the following amplification system and procedures. The amplification system was 35 μl in total: 30 μl of 1.1×T3 Super PCR Mix; 2 μl of 10 μM Primer F; 2 μl of 10 μM Primer R; and 1 μl of a Template (gDNA). Amplification procedure: 98° C. for 3 min; 98° C. for 10 s, 57° C. for 10 s, 72° C. for 15 s, 35 cycles; 72° C. for 2 min; storage at 4° C. The amplified PCR product was subjected to agarose gel electrophoresis (2 μl of the sample+6 μl of bromophenol blue) at a voltage of 300 V for 12 minutes, to acquire an identification gel photograph through which the size of a band of interest was determined. The PCR products were purified by utilizing an MagS Magnetic Bead Gel Recovery Kit (Tsingke Biotechnology Co., Ltd., Beijing).

The purified single PCR products were ready for use. The single base extension primers were diluted to 10 μM, and SNaPshot PCR was conducted. The PCR system was 5 μl in total: 2 μl of ABI SnapShot multiplex Mix (Applied Biosystems, Foster City, CA, USA); 1 μl of the Primers; 1 μl of a purified Post-PCR Template; and 1 μl of ddH₂O. Amplification procedure: 96° C. for 2 min; 96° C. for 10 s, 50° C. for 5 s, 60° C. for 30 s, 30 cycles; 60° C. for 30 s; and storage at 4° C. The SNaPshot PCR reaction products were detected by capillary electrophoresis via an ABI 3730XL DNA analyzer (Applied Biosystems, Foster City, USA).

### 1.3 Data Analysis

Data analysis of SNP loci was conducted by utilizing Gene mapper 4.1, wherein each sample was genotyped according to peaks corresponding to the SNP loci, and the resultant analysis results were a file of an Excel format and a peak map of a PDF format. The genetic diversity parameters of two groups of SNP markers were calculated by utilizing PowerMarker 3.25, wherein the genetic diversity parameters included a number of genotypes (NG), a major allele frequency (MAF), a number of alleles (NA), gene diversity (GD), expected heterozygosity (He) and polymorphic information content (PIC).

Genetic structure analysis of SNP markers was carried out on Pisum sativum L. populations by utilizing different population genetic structure analysis methods. Firstly, Bayesian cluster analysis was conducted by utilizing Structure 2.3.4. The parameters were set as follows: Length of Burnin Period=10,000, Number of MCMC Reps after Burnin=100,000, Number of Population=1-10, and Number of Iterations=10. According to the algorithm proposed by Evanno et al., the optimal population structure and population size were determined according to a Delta K (ΔK) value (the online analysis website was http://taylor0.biology.ucla.edu/struct_harvest/). Secondly, principal coordinate analysis (PCoA) was conducted by utilizing GenAlEx 6.5 to check whether the population genetic analysis of the Pisum sativum L. was reasonable. Finally, a phylogenetic tree of the Pisum sativum L. population was constructed based on UPGMA (unweighted pair-group method) by utilizing PowerMarker 3.25, with and displayed Figtree 1.4.3 (https://github.com/rambaut/figtree/releases/tag/v1.4.3).

### 2. Results

2.1 Genetic Diversity Analysis of Pisum sativum L. Population

Genetic diversity evaluation of the Pisum sativum L. germplasm population was conducted by utilizing 46 neutral SNaPshot markers. The total numbers of NG and NA were 140 and 94, respectively (Table 4). The means of MAF, GD, He, and PIC were 0.705, 0.371, 0.155, and 0.293, respectively (Table 4), and the ranges of them were 0.505-0.988, 0.023-0.628, 0.005-0.539, and 0.023-0.577, respectively (Table 5). According to the magnitude of the PIC value, the SNaPshot markers could be divided into high information content (PIC≥0.5), medium information content (0.25≤PIC<0.5) and low information content (PIC<0.25). According to this standard, in total there were 1 SNaPshot marker with high PIC, 34 SNaPshot markers with medium PIC and 11 SNaPshot markers with low PIC (Table 4). The analysis results of the neutral SNaPshot markers showed that the population of 432 accessions of Pisum sativum L. germplasms had relatively high genetic diversity.

TABLE 4
Summary of genetic diversity parameters of SNP markers
in Pisum sativum L. germplasm population

| Number of | Total | Total | Average | Average | Average | Average | Type of information (PIC) | | |
|---|---|---|---|---|---|---|---|---|---|
| markers | NG | NA | MAF | GD | He | PIC | Low | Medium | High |
| 46 | 140 | 94 | 0.705 | 0.371 | 0.155 | 0.293 | 11 | 34 | 1 |
| | | | | | | | (23.9%) | (73.9%) | (2.2%) |

note:
NG: the number of genotypes; NA: the number of alleles; MAF: major allele frequency; GD: gene diversity; He: expected heterozygosity; PIC: polymorphic information content, high (PIC ≥ 0.5), medium (0.25 ≤ PIC < 0.5), and low (PIC < 0.25).

TABLE 5
Genetic diversity indicators of neutral
SNaPshot markers of Pisum sativum L.

| ID | NG | NA | MAF | GD | He | PIC |
|---|---|---|---|---|---|---|
| 1 | 3 | 2 | 0.533 | 0.498 | 0.282 | 0.374 |
| 2 | 3 | 2 | 0.686 | 0.430 | 0.110 | 0.338 |
| 3 | 3 | 2 | 0.988 | 0.023 | 0.005 | 0.023 |
| 4 | 3 | 2 | 0.901 | 0.178 | 0.072 | 0.162 |
| 5 | 3 | 2 | 0.612 | 0.475 | 0.400 | 0.362 |
| 6 | 3 | 2 | 0.780 | 0.343 | 0.133 | 0.284 |
| 7 | 3 | 2 | 0.924 | 0.141 | 0.030 | 0.131 |
| 8 | 3 | 2 | 0.622 | 0.470 | 0.150 | 0.360 |
| 9 | 3 | 2 | 0.610 | 0.476 | 0.094 | 0.363 |
| 10 | 2 | 2 | 0.899 | 0.181 | 0.201 | 0.165 |
| 11 | 3 | 2 | 0.666 | 0.445 | 0.130 | 0.346 |
| 12 | 6 | 4 | 0.537 | 0.628 | 0.215 | 0.577 |
| 13 | 3 | 2 | 0.782 | 0.341 | 0.091 | 0.283 |
| 14 | 3 | 2 | 0.616 | 0.473 | 0.133 | 0.361 |
| 15 | 3 | 2 | 0.526 | 0.499 | 0.125 | 0.374 |
| 16 | 3 | 2 | 0.701 | 0.419 | 0.259 | 0.331 |
| 17 | 3 | 2 | 0.914 | 0.157 | 0.120 | 0.144 |
| 18 | 3 | 2 | 0.820 | 0.295 | 0.095 | 0.251 |
| 19 | 3 | 2 | 0.625 | 0.469 | 0.120 | 0.359 |
| 20 | 3 | 2 | 0.579 | 0.488 | 0.148 | 0.369 |
| 21 | 3 | 2 | 0.619 | 0.472 | 0.113 | 0.360 |
| 22 | 3 | 2 | 0.575 | 0.489 | 0.336 | 0.369 |
| 23 | 3 | 2 | 0.569 | 0.490 | 0.185 | 0.370 |
| 24 | 3 | 2 | 0.543 | 0.496 | 0.211 | 0.373 |
| 25 | 3 | 2 | 0.768 | 0.357 | 0.057 | 0.293 |
| 26 | 3 | 2 | 0.964 | 0.069 | 0.030 | 0.067 |
| 27 | 3 | 2 | 0.505 | 0.500 | 0.227 | 0.375 |
| 28 | 3 | 2 | 0.511 | 0.500 | 0.141 | 0.375 |
| 29 | 3 | 2 | 0.716 | 0.407 | 0.128 | 0.324 |
| 30 | 3 | 2 | 0.508 | 0.500 | 0.285 | 0.375 |
| 31 | 3 | 2 | 0.527 | 0.499 | 0.107 | 0.374 |
| 32 | 3 | 2 | 0.670 | 0.442 | 0.322 | 0.344 |
| 33 | 3 | 2 | 0.907 | 0.168 | 0.060 | 0.154 |
| 34 | 3 | 2 | 0.952 | 0.091 | 0.030 | 0.086 |
| 35 | 3 | 2 | 0.801 | 0.319 | 0.125 | 0.268 |
| 36 | 3 | 2 | 0.968 | 0.063 | 0.032 | 0.061 |
| 37 | 3 | 2 | 0.948 | 0.099 | 0.039 | 0.094 |
| 38 | 3 | 2 | 0.931 | 0.129 | 0.021 | 0.121 |
| 39 | 3 | 2 | 0.532 | 0.498 | 0.134 | 0.374 |
| 40 | 3 | 2 | 0.616 | 0.473 | 0.132 | 0.361 |
| 41 | 3 | 2 | 0.583 | 0.486 | 0.366 | 0.368 |
| 42 | 3 | 2 | 0.626 | 0.468 | 0.539 | 0.359 |
| 43 | 3 | 2 | 0.686 | 0.431 | 0.192 | 0.338 |
| 44 | 3 | 2 | 0.525 | 0.499 | 0.162 | 0.374 |
| 45 | 3 | 2 | 0.777 | 0.347 | 0.153 | 0.287 |
| 46 | 3 | 2 | 0.791 | 0.331 | 0.086 | 0.276 |
| Mean | 3.043 | 2.043 | 0.705 | 0.371 | 0.155 | 0.293 |
| Max | 6 | 4 | 0.988 | 0.628 | 0.539 | 0.577 |
| Min | 2 | 2 | 0.505 | 0.023 | 0.005 | 0.023 |

2.2 Population Genetic Structure Analysis of Pisum sativum L. Germplasms

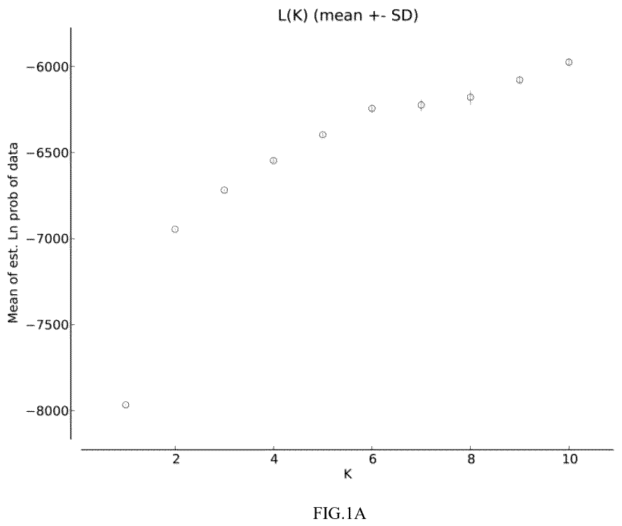

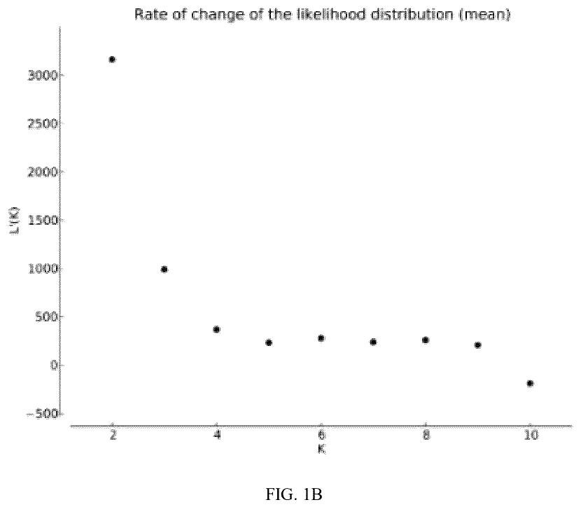

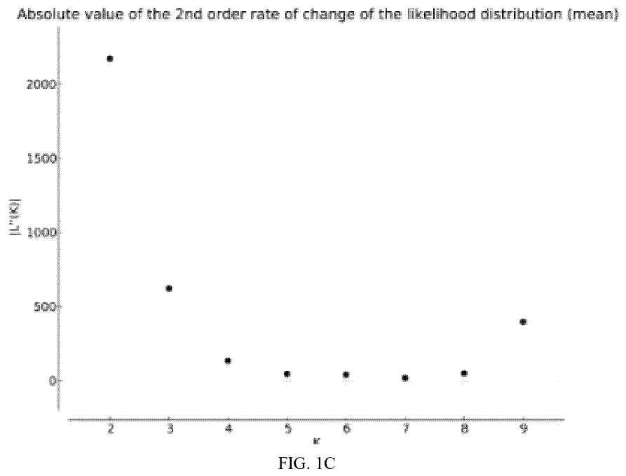

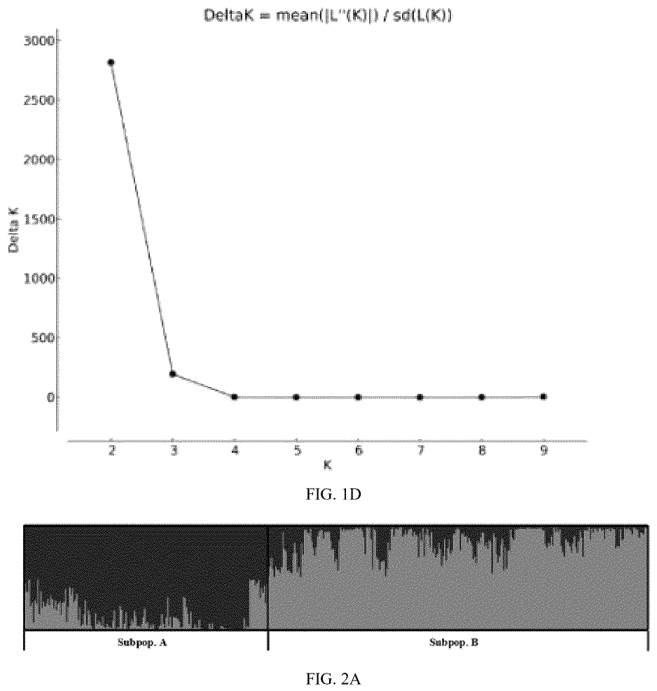

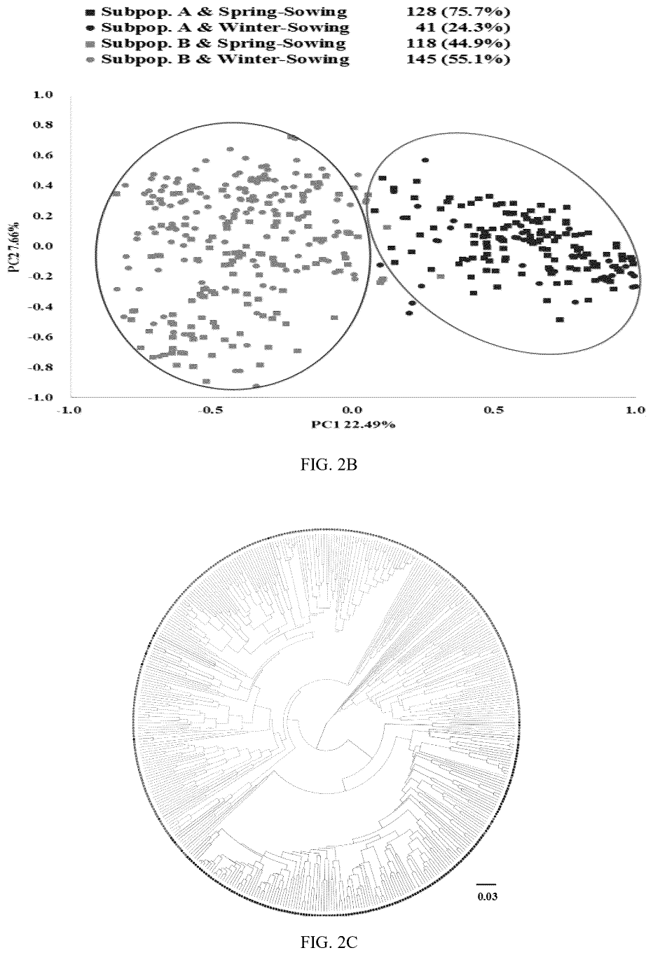

In order to study the population genetic structure of 432 accessions of Pisum sativum L. germplasms, the genetic composition of the 432 accessions of Pisum sativum L. germplasms was calculated by utilizing Structure 2.3.4, and the optimal grouping number (K) of the genetic subpopulations was determined. The Evanno' ΔK value was the highest when the grouping number K of the genetic subpopulations=2, and was much higher than other K values (FIGS. 1A-1D). In FIG. 2A, crimson (shown in black in a black-and-white diagram, the same below) represented subpopulation A, with a total of 169 accessions, including 128 accessions (75.7%) of the spring sowing type, 41 accessions (24.3%) of the winter sowing type, and the spring sowing type dominated; in the subpopulation A, 154 accessions (91.1%) were from northern China, and a few were from southern China and foreign countries, of 11 accessions (6.5%) and 4 accessions (2.4%) respectively. Green (shown in light gray in a black-and-white diagram, the same below) represented subpopulation B, with a total of 263 accessions, including 118 accessions (44.9%) of the spring sowing type, 145 accessions (55.1%) of the winter sowing type, and the number of the winter sowing type was slightly larger; in the subpopulation B, 111 accessions (42.2%) were from South China, 87 accessions (33.1%) were from North China, 57 accessions (21.7%) were from foreign countries and 8 accessions (3.0%) were from unknown sources (Table 6). The two subpopulations divided by the neutral SNaPshot markers are quite different in quantity and composition.

TABLE 6
Grouping of genetic subpopulations of Pisum sativum L. germplasms
based on Structure analysis of neutral SNaPshot markers

| | Subpop. A | | Subpop. B | | | Subpop. A | | Subpop. B | |
|---|---|---|---|---|---|---|---|---|---|
| Sowing | | Proportion | | Proportion | | | Proportion | | Proportion |
| Type | Number | (%) | Number | (%) | Source | Number | (%) | Number | (%) |
| Spring | 128 | 75.7 | 118 | 44.9 | South China | 11 | 6.5 | 111 | 42.2 |
| sowing | | | | | | | | | |
| Winter | 41 | 24.3 | 145 | 55.1 | Northern China | 154 | 91.1 | 87 | 33.1 |
| sowing | | | | | in foreign | 4 | 2.4 | 57 | 21.7 |
| | | | | | countries | | | | |
| | | | | | Unknown | — | — | 8 | 3.0 |
| Total | 169 | 100 | 263 | 100 | Total number | 169 | 100 | 263 | 100 |
| number | | | | | | | | | |

The Structure analysis Result results were verified by principal coordinate analysis (PCoA). PCoA based on the neutral markers divided the screened Pisum sativum L. germplasms into two genetic subpopulations A and B. As shown in FIG. 2B, the subpopulation A in a blue oval (the oval on the right) was clearly separated from the subpopulation B in a red oval (the oval on the left), but individual germplasm exceptions included in one subpopulation were also included in the other subpopulation, wherein a crimson square represented the subpopulation A of the spring sowing type, a crimson circle represented the subpopulation A of the winter sowing type; a green square represented the subpopulation B of the spring sowing type, and a green circle represented the subpopulation B of the winter sowing type. The population composition was consistent with that of the Structure analysis. The contribution rate of the first three components of the neutral markers in PCoA was 34.56%. The aforementioned results indicated that PCoA well validated the grouping of the genetic subpopulations of Pisum sativum L. germplasms conducted by Structure analysis.

A phylogenetic tree was constructed by utilizing UPGMA cluster analysis, and thus the analysis results could be displayed more intuitively. An UPGMA dendrogram based on the neutral markers divided all of the 432 accessions of Pisum sativum L. germplasms into two groups of tree branches. As shown in FIG. 2C, the crimson tree branch was the subpopulation A, and the green tree branch was the subpopulation B. Individual germplasms in one of the two subpopulations were also in the other one of the subpopulations, which was consistent with that of the PCoA analysis.

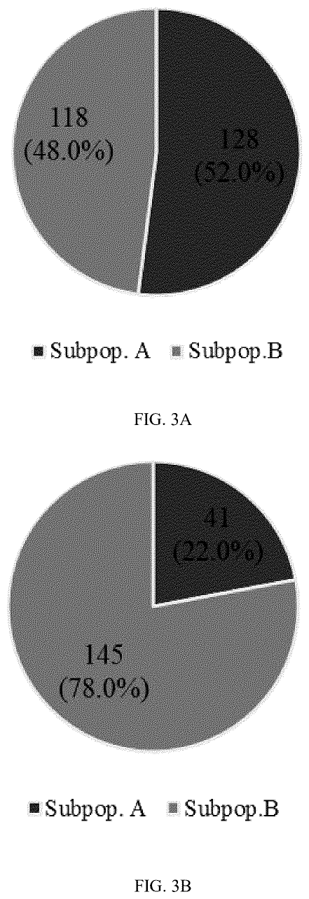

The 432 accessions of Pisum sativum L. germplasms could be divided into the spring sowing type (n=246) and the winter sowing type (n=186). 2 subpopulations were obtained through analysis of the population genetic structure of the neutral SNaPshot markers, and the genetic composition of the types of sowing dates could be resolved. As shown in FIGS. 3A-3B, among the 246 accessions of the spring sowing type, 128 accessions (52.0%) belonged to the subpopulation A, which was slightly higher than the 118 accessions (48.0%) of the subpopulation B; and among the 186 accessions of the winter sowing type, only 41 accessions (22.0%) belonged to the subpopulation A, which was much less than the 145 accessions (78.0%) of the subpopulation B, indicating that more than half of the spring sowing type belonged to the subpopulation A, while most of the winter sowing type belonged to the subpopulation B.

### 3. Discussion

In this study, the SNaPshot method was introduced into the identification and evaluation of Pisum sativum L. germplasms for the first time, and genetic diversity evaluation and population genetic structure analysis were conducted on 432 accessions of Pisum sativum L. germplasms by utilizing neutral SNaPshot markers. After analysis of the neutral SNaPshot markers, it was found that the number of the markers significantly affected the total amount of NG and NA, and had a certain impact on the means of MAF, GD and PIC, but had little impact on the mean of He. When the number of markers were increased, the total amount of NG and NA was increased, but the mean of MAF was decreased, the means of GD and PIC were increased, and the proportion of markers with high and medium PIC was increased; and vice versa. From the inside of the marker, the population size has little impact on the total amount of NG and NA, indicating that the selection of the neutral markers was scientific and the distribution on the chromosome was uniform. The population size was decreased, the mean of MAF was increased, the He did not change much, the means of GD and PIC were decreased, and accordingly the proportion of markers with high and medium PIC was decreased; and vice versa.

For the neutral markers, the Structure analysis divided the 432 accessions of Pisum sativum L. germplasms into two genetic subpopulations A and B. There were a total of 169 accessions of germplasms in the subpopulation A, of which 120 accessions (71.0%) were of the type of spring sowing in northern China, accounting for the majority; and there were a total of 263 accessions of germplasms in the subpopulation B, among which the top three were 99 accessions (37.6%) of the type of winter sowing in southern China, 60 accessions (22.8%) of the type of spring sowing in northern China and 42 accessions (16.0%) of the type of spring sowing in foreign countries. This was highly consistent with the actual production of Pisum sativum L., because the northern China belonged to the spring sowing area of Pisum sativum L., while the southern China belonged to the winter sowing area of Pisum sativum L., and most of the foreign germplasm sources are Europe and North America, which had higher latitude and lower temperature and belonged to the spring sowing area of Pisum sativum L. Principal coordinate analysis (PCoA) and UPGMA cluster analysis dendrogram could verify the results of Structure analysis more intuitively. This result was due to the fact that the neutral markers could better group the Pisum sativum L. germplasms according to their geographic origins due to their independence of functional genes such as heat tolerance, and were more consistent with a type of sowing date.

1. A set of neutral SNaPshot markers of Pisum sativum L., consisting of 46 neutral SNaPshot markers shown in the table below:

| Serial | | |
|---|---|---|
| Number | Marker Name | Sequence |
| 1 | PsCam036172_ | TTTTGCTTGTCCAATATTGTATTGTGAGTGCTGGTTTTGAACCTGAAATTTTTTG |
| | 21320_1822 | TGGTG[A/G]TCAACGATTCAGAAACATGAATTATTATTTTGTCTTTAACTTGTAT |
| | | GTTTGTCTTAGGAT |
| 2 | PsCam035943_ | CTTTTCATCAAACGAGTTCATCGAATCCAAGCTTGTGATTCTCTACGCAAAATG |
| | 21097_618 | CGGCCT[T/C]GCGGATATCGCGGTTCATCTCTTTCGTAACGTTGTTCAAAACCA |
| | | GAATCTCTTCTCTTGG |
| 3 | PsCam057738_ | CACCGAGATGGAACGTTGAAGCAAAAGGTTGCAGAGAATCCCGAGAGGTATA |
| | 38296_1700 | GATGTAAA[A/G]TCGCTAGTGTATGAGTTCGCGATGAAATTATCTGACTTGCCG |
| | | TTTTTCTTTGAAATTTAA |
| 4 | PsCam037986_ | TATATCTTAGAACCAATCATCAGCAATGTTGTTTGAATATCCTTAACTTTAGATT |
| | 23038_1304 | TCTGC[T/C]GTCGTTTGTCCTTATTTTGAATCTGTTGTTGCAGGCTCCAATGGTG |
| | | GCCTCATGTGAAAG |
| 5 | PsCam043062_ | CTTAGTTTCTAATTCATCCACATGATGATTTTTGTTAGACAATTTTTGTTTGAGA |
| | 27092_1991 | TTCTC[T/C]TTATTAAGAAAATGCTTAGTCTTAAACATGGATCTTCATGGCTATT |
| | | GTTATTTTGTGAAC |
| 6 | PsCam049876_ | AAGCACATTCAAAATTTATTTCTCAAACATCTCAAGGCAATTAGGATCAAATTC |
| | 32490_1287 | AGGCAC[T/G]GAAGTACCGAAACAAACCTGAACTGCAGCTCTATAGAAGAGTT |
| | | CCTCTCCCACTGAACTT |
| 7 | PsCam051455_ | TGTACAATCATTATAAATATAGCTATTGTGAAGAGATGTTATGCGGAAGAAAAG |
| | 33948_107 | AAAAAC[T/G]AAGCTATGATTTTATTCTACAACCTCTTTCTTGCTCAAACAAAT |
| | | AGAACTAGAGCAAGAT |
| 8 | PsCam045637_ | GGACAAGAAATCAGTTTTGATGATTTATCTCTTGAAGAGAAGAAACAATTTCA |
| | 29281_840 | AAGAGCT[A/G]TTGCTTGTGGGGAATTAAGCAAGATGATCACACCATGGGATC |
| | | CATGGTGGTCAAAGCATT |
| 9 | PsCam025478_ | AGTTAGACTTTGGCTAATTGGCATTGCTTCTGTCTTTTTTCTGTTTCAGTTTCCA |
| | 14590_1802 | ATTGC[A/G]CTTTTGGTATTCGGCTTTGAAGCAGTAAAGTTGTACAAGGATCAC |
| | | AGAAAGAGGAAGGGC |
| 10 | PsCam041441_ | ACGTCAAGGCAGCGAGAGATATGGTGTCATCCGAAAGCCGAAACTCTACGAG |
| | 25895_460 | AGGTGAAA[T/C]TTCGTTGAAGTAAACTTCTACCTTAATCAAAAATTGAGGAA |
| | | GAAATATTTTTTGAGATGT |
| 11 | PsCam050369_ | AACTTTCAGGGCGAGGTGATTCATAACTGGAACTTTCAGAGGTAGGTGATTCA |
| | 32956_339 | TACAAAC[T/C]GGCACTTTCAGAGCTTTCAAAATCAGGAACTTCGCCATCAAA |
| | | GGAAGTATCAGGAGCATC |
| 12 | PsCam040267_ | CATCATGTGCTCAACTTTGGTCACATTTTCCACGTTTCTCAGGTGCTAAGAAGT |
| | 25050_210 | TCAAAA[T/C]CAAATAGTCTTCCCTCCATCTCCTCTTCCGCGCCGAAGGTCGCC |
| | | TCTGGCGGCGGCGGAC |
| 13 | PsCam000362_ | CGTATTTTGGTAACACACCACCAAATCAGAGAGTCTCGATGCTGAGATTCCGG |
| | 321_595 | AATCAAC[A/G]CACGCTGTTTGTAGATCTTTGCATAAGAAACGTCGAGGATTTA |
| | | ACTCGAAATAATCGAAT |
| 14 | PsCam034276_ | AAATCCACATTAGGTCTCTCTACTTTCATGTCCATATCCAAATCAGAACCCCTC |
| | 19685_1661 | CCCTCA[T/C]ACACATTGTCCTTATTCAACCAAGATCTTTCATCCAAACCTTCAT |
| | | ACTGGCAAGAAACAT |
| 15 | PsCam000088_ | GAGGAATTATGGAGAGACAATGAGACATGGAGAAAGACAGCTACAAAACAA |
| | 75_1456 | AAAACAAAA[A/G]TGCAATTGAACTTCACTATGATACTGTTTTTGTTTCTTACA |
| | | TGGTTGTCTGTTTTTGTGT |
| 16 | PsCam037526_ | TTCTTCTTCTTCAAGCTGCTTAAGCCAAAGAATGGCATGAACCCTCTGTCTCAT |
| | 22605_590 | AATCCT[A/G]TAATCCTTAGCAAGCTTCTCAACAGTATAAACCTCAGGATCCTT |
| | | TTTGTGCAAGCGATAC |
| 17 | PsCam055871_ | GCTTCCAGAATAGCTTGATTAGCGGTGAAGTGAGCGAATTTGAGATAAGGAGA |
| | 36784_1179 | AGACTCG[T/G]AAAAGTGCATGTGAAGTGCATCAGACAAAGATGAATCCAGA |
| | | GTTTCATCAGGGGAAACTC |
| 18 | PsCam043018_ | AGCCACTAATTGACAATTAGACATAGTTGTATATGTATATATGTTAGTGAGGACA |
| | 27052_1394 | ATAAG[A/G]AAAGAAAATCAGATTGTATATACATTAAGCGTGGTGAAGAAATAT |
| | | ATGTGAAAAAGCCTT |
| 19 | PsCam040468_ | CTAATTTAATCAACTTTCATTCAAAAGCAAAACACCTTGCAGCATAGCACAAC |
| | 25210_90 | TGCAAGA[A/C]GAGCACAATGGCACTATATACATAAGAGAGGTTTTAATCACCC |
| | | TAAACAACCAAAAAAGA |
| 20 | PsCam036980_ | AGAATGCAATCTGACGAAAGCCATGAGGCACGAAGTTTATACCATCATCCACT |
| | 22091_68 | AAAAACA[T/C]GCCACGTAATCATGCCAAAACTCCGTCCAGGTTCAATAAACC |
| | | GCCACAATAGAGAAACCT |
| 21 | PsCam004622_ | CATGGATCCTCTGGAGTCTTTCTTCGCGTCCTGACTTTTTAACGAGTGAAATAG |
| | 3470_1686 | TTGGAC[A/G]GTTGCAGAAGCCTTATGAGTGTTGGCCAATTTTGCCAAAGGCC |
| | | GAGACGGAATCGGCGGT |
| 22 | PsCam035376_ | CTGATACACCTTACCCTCCGCAAAAGTCAAACAAAGATGCTTCCCAGAAAGA |
| | 20564_2643 | AAATGTTA[A/G]TGAATATGCAAAACATGACATCATAAGTGGTGAAAGCAACA |
| | | GTGTGGTGTCACTGAAACA |
| 23 | PsCam044939_ | CATGTCATAGGGGCCTAGACCGTAACCCATCATATGCATTGGACCAGCAGCAG |
| | 28681_1017 | CATATGG[T/G]GCCATAAATCCATCCATACCAGGTTGGATACCATTCCAGTATGG |
| | | GTTATAACCAGGAGGT |
| 24 | PsCam005290_ | ACAGAAAGTCAAGATCGCAGAAAAGATATTCCCGATGATAACTGAGACGACG |
| | 4012_1343 | AGGACACC[A/G]ACCCATTTGACCCTCTCAACGAAATTATCAAAAAATTCCAG |
| | | TAAAGAGAATTGATTTGCC |
| 25 | PsCam026873_ | TTGAGAACACTTGCAATTTCAACAATCACATGATAATCTCTGGAAACATTCTTA |
| | 15607_1235 | AGAGCT[A/G]CATTTGCCAACACAGCATAACAATCTGAAGGTTCCAATATCCAC |
| | | CTATACATTGCTTTCT |
| 26 | PsCam042409_ | TTTAATCTTATATATACATACATGAAGGAAAAAAAATATAGAATCAAAAAATTGT |
| | 26473_1566 | ACCAT[T/C]AGAAGAAGCAGATAAGGTTGGAATTTGTGAGCTTGGAAACCAAG |
| | | CAAAGAATTATCCTAA |
| 27 | PsCam043345_ | CACAGCGCAACGACCGTGGGACCCACCTCTCGTTCAAGAGGGTCGGGTTCGG |
| | 27360_370 | TCCTTCTG[T/C]ACGGGTCGGAAGTTTCGCAATCTTTTCAAATATCCGCTTCATC |
| | | GTGTCTTCGTTTCACCG |
| 28 | PsCam006884_ | GGAATCATGACAGGTTCGATGGAACCGTATTTGGAAAAACTGAGACACTATGC |
| | 5125_2126 | AGGTGTG[A/G]TACCTTTGTTGACCGCTGATTACGGAGCCTCTGAAGGATGGAT |
| | | AGCTGCAAATGTGAATC |
| 29 | PsCam057416_ | AAAAAAATAACAGACTCAAACTCTATCCAATGGGATATTAAAGCAATACGCAA |
| | 38023_271 | CCAAAGA[T/C]GGAAGCAGGCACAAACGACCAGCATTTTTGGAAAGTGCACT |
| | | CTTGTGGCAAATGATTGCG |
| 30 | PsCam004972_ | ATTTCCTTATTTATGTGTCAAAAGATCGATCGCGCAAAATAAAATAAAATACAA |
| | 3765_1941 | ATCTCC[A/G]CCGATACAATGTATTTTCTTTTCCACAAACAAAACAAAACAAAA |
| | | GTTACCATTTTCTTCT |
| 31 | PsCam049238_ | AATCATCAACAAAAATACATAGCGAGACTTCTCTATACTCTTTTATGATTTTCGA |
| | 31877_3224 | ATATG[A/G]ACCATAACTACAAAATAAACCACAAGTGGCAATGCAGTAATGCAT |
| | | AAATACCTGTTTGAC |
| 32 | PsCam049156_ | TTTTGGAGCCAAGGATGCTCTGCAAGACAAGTAAAGGAAAACATCATGAAGT |
| | 31799_1877 | CAGTGAAT[T/C]TATTCTGATAAATCCTTAACATAAACAGCATAAATGATTTCTA |
| | | ATCATTATTTATTTCAG |
| 33 | PsCam054451_ | AGCATGGCCATCCCCAAAAAGGGAACGAGAGCTGAGAGCAGAACGGCCGAT |
| | 35939_483 | AATTAGTGA[T/C]GCGGACACTAAAACATTATCAAAATTCATGAAGTGTTCATA |
| | | TTAAGAAATAAAAATCATA |
| 34 | PsCam034709_ | GTGTTTGAAGTTCCGATTGAAAAGTTGGAAATCGAATTCAGAAAGCCTGTAGA |
| | 20077_714 | TCAGTTA[A/G]AGAAGGAAACAATATCGGAGTCAGAGAAGCCTTTTGTGAACG |
| | | AACTGACAATGAAAAACG |
| 35 | PsCam055500_ | TTTGTTTTCCACGAAAGAACTAAACACATTAAAGTGGATTGTCATTTCATAAG |
| | 36558_381 | AGACCAT[A/G]TTTAATCAGGTGTCATCACTATGACTCATGTTTCAACTCATCA |
| | | GCAATAGGCTGACATCT |
| 36 | PsCam033926_ | GGTTTATAGTCGTCCAAAAACTAAGGTAGCGGTAGTACCGTCAGAAATAGAGC |
| | 19423_1600 | AGAAGAG[T/C]GGTGAAAGTGATGGTGAATATATTAAGCGGCTGAAAGCTTTT |
| | | GTTGAGAATCAGCACTCT |
| 37 | PsCam051338_ | ACAAGCACAGGAAAGGTTACTAATCATACCTATTTCTAAGTGTGGCATAGTTAG |
| | 33847_320 | AATGCA[A/G]AAAGAGTAGTACGATCAAAAGCAGCAAGACATCAGAACAAAT |
| | | TGTTAAGAATGGTAAATC |
| 38 | PsCam037094_ | GGAACATTTTGTCTGTCTAGGACCTTTGTAGCACCGACACGTCTGAATTTTTTT |
| | 22201_1066 | TTGGAA[T/C]GATTGGATTGAATGACTTAATCAGAGTATGAGGATCATTCCTGT |
| | | AGTACCTTTTTTCAAC |
| 39 | PsCam004372_ | TGAAGGAGTTGGACAATTATTCGTTGGTGTCGAATACGAGGGAATGACTTGAG |
| | 3289_2443 | GAGCTTT[A/G]CAGTAAACCTCATTAGAAACTCCATTTTCAAGACAAATCTTGT |
| | | TTTGACCCAATCTGCAA |
| 40 | PsCam014062_ | ATGTAAAGACTATGGAAGAGCTTACCTGATCAATTTTACTTCGAAAAATACGTT |
| | 9583_426 | TCAAGA[T/G]CCTGTTAAAGTGTTTGTTTTGGTGAATCTTCCCAATTTCGTCGT |
| | | GCAGTTTTTCTAGCAA |
| 41 | PsCam010804_ | AACTCCTTCCTAAGTTCTGAGAAAGTTTAAGTTATGAAAAATATAATGTTTAAG |
| | 7280_996 | GCCTTA[A/G]TGATGACAATTACCCAATAATAAGATAAGTTGAATGCATATGATG |
| | | CAGGTACTCTGACGG |
| 42 | PsCam040802_ | ATCTGTAAATTCAGCAACACACATTGTTGGAGGCAAGGTTGGTTGGAATTTGC |
| | 25456_306 | CTAGTTA[T/C]TATAGTTTCTTTGAGGATTGGTCAAAGAACCAAACCTTCATTG |
| | | TTGGCGATCAACTTCGT |
| 43 | PsCam001376_ | TGTTGTCACTATCAAAGACAAATTTGATGATAGTGAGATAGGAAAGGAATAAA |
| | 1148_333 | ACACAGT[A/G]TTTAGATCATAGATAATATTTGTACACTTAAAGTTTTAACATTT |
| | | GTGACACCTAGTTCAA |
| 44 | PsCam000349_ | CAAATGACCCATTCAATCATTTTCTGTTGGCCATGTTGCCTAAGCCTTGTTTGG |
| | 309_200 | CAGTGT[T/C]GGGATACTTTGTTCATTCCAGAAAAAGTTTTGAGGATCAACTTG |
| | | AGTCTTAACCTTATTT |
| 45 | PsCam037467_ | TCTAAAATACATTCCTGATGGAAACTACATAAAGGTTGGAAGCGTCGCCACAA |
| | 22549_557 | TCAACAA[A/G]CCAGACTTGTTGCCAACACTCTCCACATTGCGTTACTTTACCA |
| | | ACACGTTATCTAAAAAA |
| 46 | PsCam024028_ | CCTGTTCTTCACATTGAATCCCTTGGTCATGCCCTTCATGCTTTTATAAACGGG |
| | 13660_713 | AAACTC[A/G]CAGGTATCTTTCTTTACTTCAATGAAGAATCTCATGAATCATGTA |
| | | ATGATATATCGATAT |

2. The set of neutral SNaPshot markers of Pisum sativum L. according to claim 1, wherein peripheral amplification primer sequences and single base extension primer sequences for the 46 neutral SNaPshot markers are shown in the table below:

| | | Names of | | | |
|---|---|---|---|---|---|
| | | peri- | | Names of | |
| | | pheral | Sequences of | SNP single | |
| | | ampli | peripheral | base | Sequences of SNP |
| Serial | | fication | amplification | extension | single base |
| Number | Marker Name | primers | primers | primers | extension primers |
| 1 | PsCam036172_ | 1-F | TCCTAAACACAGCAC | 1-SNP-F | TTTTTTTTTTACCTGAAATTT |
| | 21320_ | | TCAACAC | | TTTGTGGTG |
| | 1822 | 1-R | ACCCCACCAGATTGA | | |
| | | | GATGA | | |
| 2 | PsCam035943_ | 2-F | CCCCAATCCCCTCCA | 2-SNP-F | TTTTTTTTTTTTTTTTTTTACG |
| | 21097_618 | | AAACA | | CAAAATGCGGCCT |
| | | 2-R | GAGAGTCCCATTCGG | | |
| | | | GCTTG | | |
| 3 | PsCam057738_ | 3-F | GCCGAAGCCTACTTG | 3-SNP-F | TTTTTTTTTTTTTTTTTTTCCC |
| | 38296_1700 | | TTTGC | | GAGAGGTATAGATGTAAA |
| | | 3-R | GCATTTCAAGCATGG | | |
| | | | GGACT | | |
| 4 | PsCam037986_ | 4-F | TGAGGTTGACGACTG | 4-SNP-F | TTTTTTTTTTTTTTTTTTTTTT |
| | 23038_1304 | | CCTTT | | TTTTTTTATATCCTTAACTTTA |
| | | 4-R | GCGCAGCACCAAAAT | | GATTTCTGC |
| | | | AAGGTA | | |
| 5 | PsCam043062_ | 5-F | CCGAGGCAGAGAAG | 5-SNP-R | TTTTTTTTTTTTTTTTTTTTTT |
| | 27092_1991 | | GAAGAC | | TTTTTTTTTTTTAAGACTAAG |
| | | 5-R | TGTGCCCTTCATAAC | | CATTTTCTTAATAA |
| | | | CACTGAT | | |
| 6 | PsCam049876_ | 6-F | ACGCCATTCCTCGAA | 6-SNP-F | TTTTTTTTTTTTTTTTTTTTTT |
| | 32490_1287 | | CATCT | | TTTTTTTTTTTTTTTTTTATTA |
| | | 6-R | GGCATGTCTCCTTTG | | GGATCAAATTCAGGCAC |
| | | | CAGGT | | |
| 7 | PsCam051455_ | 7-F | GGTTCTGCATTTCCA | 7-SNP-R | TTTTTTTTTTTTTTTTTTTTTT |
| | 33948_107 | | AATCAACT | | TTTTTTTTTTTTTTTTTTAGGT |
| | | 7-R | TCACTTCCACCTCTTT | | TGTAGAATAAAATCATAGCTT |
| | | | ATCCGC | | |
| 8 | PsCam045637_ | 8-F | TGGTTGGGTGAGGCT | 8-SNP-F | TTTTTTTTTTTTTTTTTTTTTT |
| | 29281_840 | | GATTT | | TTTTAGAAGAAACAATTTCA |
| | | 8-R | CCGTTGTAGAGGCGA | | AAGAGCT |
| | | | AGAGT | | |
| 9 | PsCam025478_ | 9-F | TGGTATTGGGTGCTC | 9-SNP-F | TTTTTTTTTTCTGTTTCAGTTT |
| | 14590_1802 | | TTCGG | | CCAATTGC |
| | | 9-R | ATAAATCCGCCTCCC | | |
| | | | GAACC | | |
| 10 | PsCam041441_ | 10-F | GGTTGTATTTCACTCT | 10-SNP-F | TTTTTTTTTTTTTTTTCGAAA |
| | 25895_460 | | CCCGTT | | CTCTACGAGAGGTGAAA |
| | | 10-R | ATAGAGGGGTTGTGG | | |
| | | | GTCAAG | | |
| 11 | PsCam050369_ | 11-F | TTGGATTCTTTACTGG | 11-SNP-F | TTTTTTTTTTTTTTTTTTTTTC |
| | 32956_339 | | GAGCTG | | AGAGGTAGGTGATTCATACA |
| | | 11-R | TGGGTTGGTGGCAGA | | AAC |
| | | | ATAGG | | |
| 12 | PsCam040267_ | 12-F | GAAACAGGGAGGTG | 12-SNP-F | TTTTTTTTTTTTTTTTTTTTTT |
| | 2505_210 | | TGGGAG | | TTTTTTTTTTCAGGTGCTAAG |
| | | 12-R | TGGATTGACGTGGTT | | AAGTTCAAAA |
| | | | GTTTGG | | |
| 13 | PsCam000362_ | 13-F | TTATCGCTAGCAGGG | 13-SNP-F | TTTTTTTTTTTTTTTTTTTTTC |
| | 321_595 | | GACAAC | | TGAGATTCCGGAATCAAC |
| | | 13-R | GATACCGAGATACAG | | |
| | | | TTCCTGC | | |
| 14 | PsCam034276_ | 14-F | TCCTCAATTCCCTTC | 14-SNP-F | TTTTTTTTTTTTTTTTTTTTTT |
| | 19685_1661 | | AGTGCC | | TTTTTTTTTTTTTTTTTTTTTT |
| | | 14-R | TGGTAATGGAGGAGG | | CAGAACCCCTCCCCTCA |
| | | | CGAGA | | |
| 15 | PsCam000088_ | 15-F | GTCTGAAACCGACAC | 15-SNP-R | TTTTTTTTTTTTTTTTTTTTTT |
| | 75_ | | CGACA | | TTTTTTTTTTTTTTTTTTAGTA |
| | 1456 | 15-R | CTGTCCAAGGAACA | | TCATAGTGAAGTTCAATTGC |
| | | | GCCTCA | | A |
| 16 | PsCam037526_ | 16-F | ACTCAGGGAAAGTG | 16-SNP-F | TTTTTTTTTTTTTTTTTTTTTT |
| | 22605_590 | | TCGAGC | | TTTTTTTTTTTTTGAACCCTC |
| | | 16-R | TGGAAGAAACGAAT | | TGTCTCATAATCCT |
| | | | GGCGGA | | |
| 17 | PsCam055871_ | 17-F | GCCACTGCATCCCTT | 17-SNP-F | TTTTTTTTTTTGAGATAAGGA |
| | 36784_1179 | | GTTTG | | GAAGACTCG |
| | | 17-R | TCACAGGAAACTGG | | |
| | | | GGTTCG | | |
| 18 | PsCam043018_ | 18-F | ATGGGATCAGCTTCG | 18-SNP-F | TTTTTTTTTTTTTTTTGTATAT |
| | 27052_1394 | | GCTTC | | ATGTTAGTGAGGACAATAAG |
| | | 18-R | ACAAAGAAGAAGGG | | |
| | | | CGGTGG | | |
| 19 | PsCam040468_ | 19-F | TGAACCAGTAACACA | 19-SNP-F | TTTTTTTTTTTTTTTAGCATAG |
| | 25210_90 | | AAACTACCA | | CACAACTGCAAGA |
| | | 19-R | CTTTGGCAGCAGGAG | | |
| | | | TGAGA | | |
| 20 | PsCam036980_ | 20-F | GCCTGACGGAAGAA | 20-SNP-F | TTTTTTTTTTTTTTTTTTTTTT |
| | 22091_68 | | CGGAA | | TTTTTATACCATCATCCACTA |
| | | 20-R | AAGAAACAGCGAGA | | AAAACA |
| | | | GCCATGA | | |
| 21 | PsCam004622_ | 21-F | GCTAACAGATGAGCC | 21-SNP-F | TTTTTTTTTTTTTTTTTTTTTT |
| | 3470_1686 | | GAACG | | TTTTTTTTTTTTTTTTTTTAAC |
| | | 21-R | ACCAATCAATGCCTC | | GAGTGAAATAGTTGGAC |
| | | | CTGCC | | |
| 22 | PsCam035376_ | 22-F | GAGCCGTTTGATTGC | 22-SNP-R | TTTTTTTTTTTTTTTTTTTTTT |
| | 20564_2643 | | TGGTG | | TTTTTTTTTTTTTGATGTCATG |
| | | 22-R | ACTGATCCTGTGGCA | | TTTTGCATATTCA |
| | | | AACCA | | |
| 23 | PsCam044939_ | 23-F | GAGGCGGAGGAGGA | 23-SNP-F | TTTTTTTTTTTTTTTTTTTTTT |
| | 28681_1017 | | ATAGGA | | ACCAGCAGCAGCATATGG |
| | | 23-R | TTTCAGTGGAAACCC | | |
| | | | CCACA | | |
| 24 | PsCam005290_ | 24-F | CCAACACAACACATT | 24-SNP-F | TTTTTTTTTTTTTTTTTTTTTT |
| | 4012_1343 | | AGAAGCTCA | | TTTTTTTTTTTTTTTTTTTTTT |
| | | 24-R | TGCTGGTTGGTTGTT | | TTCTGAGACGACGAGGACAC |
| | | | TGGTAG | | C |
| 25 | PsCam026873_ | 25-F | CACATTCACAAGCTC | 25-SNP-F | TTTTTTTCTCTGGAAACATTC |
| | 15607_1235 | | TTCAGGT | | TTAAGAGCT |
| | | 25-R | GGTGGGGAACCGAT | | |
| | | | GAGAG | | |
| 26 | PsCam042409_ | 26-F | CAACAACCACTTCCA | 26-SNP-R | TTTTTTTTTTTTTTTTTTTTTT |
| | 26473_1566 | | CAGATACA | | TTTTTTTTTTTTTTTTTTCCAA |
| | | 26-R | TTCCATCCAACTCAC | | CCTTATCTGCTTCTTCT |
| | | | ACATCTC | | |
| 27 | PsCam043345_ | 27-F | ACTTCGATCCGCTAT | 27-SNP-F | TTTTTTTTTTTTTTTTTTCGGG |
| | 27360_370 | | GGACAC | | TTCGGTCCTTCTG |
| | | 27-R | GTGCTTCGGGGCTAT | | |
| | | | CATCG | | |
| 28 | PsCam006884_ | 28-F | TCTTACCAGCAGAGT | 28-SNP-F | TTTTTTTTTTTTTTTTTTACTG |
| | 5125_2126 | | CACGG | | AGACACTATGCAGGTGTG |
| | | 28-R | TCTCAAGCTGCGAAA | | |
| | | | GAGGAA | | |
| 29 | PsCam057416_ | 29-F | AGCCTCTAGGTATCC | 29-SNP-F | TTTTTTTTTTTTTTTTTTTTTT |
| | 38023_271 | | AGGCA | | TTTTAGCAATACGCAACCAA |
| | | 29-R | AGAGCTTATCGGCTT | | AGA |
| | | | ACAGCAT | | |
| 30 | PsCam004972_ | 30-F | AACGCCCGAAACTC | 30-SNP-F | TTTTTTTTTTTTTTTTTTTTTT |
| | 3765_1941 | | AAATGG | | TTTTCAAAATAAAATAAAATA |
| | | 30-R | AGGTTGCCAGTGAA | | CAAATCTCC |
| | | | AGGAGA | | |
| 31 | PsCam049238_ | 31-F | AGACAGCAGGTGTTC | 31-SNP-F | TTTTTTTTTTTTTTTTTTTTTT |
| | 31877_3224 | | GTTGT | | TTTTTTTTTATACTCTTTTATG |
| | | 31-R | GCCCCTCAACGTGTC | | ATTTTCGAATATG |
| | | | TTTGT | | |
| 32 | PsCam049156_ | 32-F | AAGCCTTGACTTGAC | 32-SNP-F | TTTTTTTTTTTTTTTTTTTTTT |
| | 31799_1877 | | GACATCT | | TTTTTTTGAAAACATCATGAA |
| | | 32-R | TGAATGGTTGAAGGA | | GTCAGTGAAT |
| | | | GAAGGGT | | |
| 33 | PsCam054451_ | 33-F | ATATCAATCTCGGATA | 33-SNP-F | TTTTTTTTTTTTTTTTTTTTGA |
| | 35939_483 | | GCAGCAC | | ACGGCCGATAATTAGTGA |
| | | 33-R | CCGTTCCTTCACAGA | | |
| | | | TGGGT | | |
| 34 | PsCam034709_ | 34-F | CCAGGCACAGCAAG | 34-SNP-F | TTTTTTTTTTTTTCAGAAAGC |
| | 20077_714 | | AGTTGA | | CTGTAGATCAGTTA |
| | | 34-R | CAAACTCGATTTCAA | | |
| | | | CGACGC | | |
| 35 | PsCam055500_ | 35-F | TCCATGCACATTTCCT | 35-SNP-F | TTTTTTTTTTTTTTTTTTTTTG |
| | 36558_381 | | ACACCT | | ATTGTCATTTCATAAGAGACC |
| | | 35-R | CCCCCTTAAGTTGGA | | AT |
| | | | GAGTGA | | |
| 36 | PsCam033926_ | 36-F | CTGTCAAAAGGCTGG | 36-SNP-F | TTTTTTTTTTTTTTTTTTTTTT |
| | 19423_1600 | | AGGCA | | TTTTTCGTCAGAAATAGAGC |
| | | 36-R | ACAAAAGCGACAAC | | AGAAGAG |
| | | | CAAAAACGA | | |
| 37 | PsCam051338_ | 37-F | TGTTGGTGGTTGTCT | 37-SNP-F | TTTTTTTTTTTTTTTTTTTTTT |
| | 33847_320 | | GCTCA | | TTTTTTTTTAGTGTGGCATAG |
| | | 37-R | GTTTCGTTCGCTGCC | | TTAGAATGCA |
| | | | ATTGT | | |
| 38 | PsCam037094_ | 38-F | AGCGAAGAGGATGA | 38-SNP-F | TTTTTTTTTTTTTTTTTTTTTT |
| | 22201_1066 | | CATGAGTA | | TTTTTTTTTTTTTTCGTCTGAA |
| | | 38-R | TGCTTCGTCTGTTTC | | TTTTTTTTTGGAA |
| | | | GGGAG | | |
| 39 | PsCam004372_ | 39-F | GCGCATTTACAGTTT | 39-SNP-F | TTTTTTTTTTTTTTTTTTTTTT |
| | 3289_2443 | | GGGCT | | TTTTTTTTTTTTTTTTTTTGGA |
| | | 39-R | CGACCTCGAGATGGG | | ATGACTTGAGGAGCTTT |
| | | | AAACC | | |
| 40 | PsCam014062_ | 40-F | TGCGACGTAATTGCT | 40-SNP-F | TTTTTTTTTTTTTTTTTTTTTT |
| | 9583_426 | | CAAAGT | | TTTTTTTTTTTCTTCGAAAAA |
| | | 40-R | AGGCTTTCGGAGGA | | TACGTTTCAAGA |
| | | | AAACAGA | | |
| 41 | PsCam010804_ | 41-F | GGTGAACCCTTGGCA | 41-SNP-R | TTTTTCTTATTATTGGGTAATT |
| | 7280_996 | | ACTTC | | GTCATCA |
| | | 41-R | ATGGTCGCTTCCCAC | | |
| | | | TTTCT | | |
| 42 | PsCam040802_ | 42-F | TGGCTGAGAAAGTG | 42-SNP-F | TTTTTTTTTTTTTTTTTTTGGT |
| | 25456_306 | | AACCTTAGT | | TGGAATTTGCCTAGTTA |
| | | 42-R | TGGTGTGTGTCGGTG | | |
| | | | GAAA | | |
| 43 | PsCam001376_ | 43-F | ATAGACAACTAGAGA | 43-SNP-F | TTTTTTTTTTTTTTTTGATAGG |
| | 1148_333 | | TTGGTTTTTGAAG | | AAAGGAATAAAACACAGT |
| | | 43-R | GGTTAACAATGTCAA | | |
| | | | TGTACACAATCA | | |
| 44 | PsCam000349_ | 44-F | AGGGCCAGAAGAAG | 44-SNP-F | TTTTTTTTTTTTTTTTTTTTTT |
| | 309_200 | | TAACAAAAG | | TTTTAAGCCTTGTTTGGCAGT |
| | | 44-R | TTGGGAAGGATCAGA | | GT |
| | | | AGCTGG | | |
| 45 | PsCam037467_ | 45-F | CTGTGGAGGCACAAA | 45-SNP-R | TTTTTTTTTTTTTTTTTTTTTT |
| | 22549_557 | | TGAGGT | | TTTTTTTTTTCGTCGCCACAA |
| | | 45-R | CACGCTCAACCTCTT | | TCAACAA |
| | | | CCCAT | | |
| 46 | PsCam024028_ | 46-F | ACACGACGGCAGATA | 46-SNP-F | TTTTTTTTTTTTTTTTTTTTTT |
| | 13660_713 | | AAAGTG | | TTTTTTTTTTTTTTTTTGCTTT |
| | | 46-F | GCGTTTCCGCTGTT | | TATAAACGGGAAACTC |
| | | | TCCTAC | | |

3. Use of the set of neutral SNaPshot markers of Pisum sativum L. according to claim 1 in analysis of genetic diversity of a Pisum sativum L. population.

4. Use of the set of neutral SNaPshot markers of Pisum sativum L. according to claim 2 in analysis of genetic diversity of a Pisum sativum L. population.

5. Use of the set of neutral SNaPshot markers of Pisum sativum L. according to claim 1 in analysis of a genetic structure of a Pisum sativum L. population.

6. Use of the set of neutral SNaPshot markers of Pisum sativum L. according to claim 2 in analysis of a genetic structure of a Pisum sativum L. population.

7. A method for analyzing genetic diversity of Pisum sativum L. by employing the set of neutral SNaPshot markers of Pisum sativum L. according to claim 2, comprising:
1) SNaPshot PCR reaction
conducting peripheral amplification by using DNAs of the population of Pisum sativum L. germplasms to be tested as PCR templates with each locus being subjected to single amplification, purifying PCR products and then conducting SNaPshot PCR of them by employing single base extension primers, and detecting reaction products of the SNaPshot PCR by capillary electrophoresis via an ABI 3730XL DNA analyzer; and
2) data analysis
conducting data analysis of SNP loci by utilizing Gene mapper 4.1, wherein each sample is genotyped according to peaks corresponding to the SNP loci, and the resultant analysis results are a file of an Excel format and a peak map of a PDF format, and calculating genetic diversity parameters of two groups of SNP markers by utilizing PowerMarker 3.25.

8. The method for analyzing genetic diversity of Pisum sativum L. according to claim 5, wherein the genetic diversity parameters of the two groups of SNP markers comprises a number of genotypes NG, a major allele frequency MAF, a number of alleles NA, gene diversity GD, expected heterozygosity He and polymorphic information content PIC.

9. The method for analyzing genetic diversity of Pisum sativum L. according to claim 7, wherein an amplification system for the peripheral amplification is 35 μl in total: 30 μl of 1.1×T3 Super PCR Mix; 2 μl of 10 μM Primer F; 2 μl of 10 μM Primer R; 1 μl of a Template; and an amplification procedure: 98° C. for 3 min; 98° C. for 10 s, 57° C. for 10 s, 72° C. for 15 s, 35 cycles; 72° C. for 2 min; and storage at 4° C.

10. The method for analyzing genetic diversity of Pisum sativum L. according to claim 8, wherein an amplification system for the peripheral amplification is 35 μl in total: 30 μl of 1.1×T3 Super PCR Mix; 2 μl of 10 μM Primer F; 2 μl of 10 μM Primer R; 1 μl of a Template; and an amplification procedure: 98° C. for 3 min; 98° C. for 10 s, 57° C. for 10 s, 72° C. for 15 s, 35 cycles; 72° C. for 2 min; and storage at 4° C.

11. The method for analyzing genetic diversity of Pisum sativum L. according to claim 7, wherein SNaPshot PCR is conducted with the single-base extension primers, and the PCR system is 5 μl in total: 2 μl of ABI SnapShot multiplex Mix; 1 μl of Primers; 1 μl of purified PCR Template; 1 μl of ddH₂O; and an amplification procedure: 96° C. for 2 min; 96° C. for 10 s, 50° C. for 5 s, 60° C. for 30 s, 30 cycles; 60° C. for 30 s; and storage at 4° C.

12. The method for analyzing genetic diversity of Pisum sativum L. according to claim 8, wherein SNaPshot PCR is conducted with the single-base extension primers, and the PCR system is 5 μl in total: 2 μl of ABI SnapShot multiplex Mix; 1 μl of Primers; 1 μl of purified PCR Template; 1 μl of ddH₂O; and an amplification procedure: 96° C. for 2 min; 96° C. for 10 s, 50° C. for 5 s, 60° C. for 30 s, 30 cycles; 60° C. for 30 s; and storage at 4° C.

13. A method for analyzing a genetic structure of a Pisum sativum L. population by employing neutral SNaPshot markers of Pisum sativum L., comprising, on the basis of step 2) of claim 5, firstly conducting Bayesian cluster analysis by utilizing Structure 2.3.4, and determining an optimal population structure and population size according to a ΔK value; secondly, conducting principal coordinate analysis PCoA to check whether a result of the Structure analysis of Pisum sativum L. is reasonable; and finally constructing a phylogenetic tree by utilizing UPGMA cluster analysis to display the analysis result intuitively.

14. The method for analyzing a genetic structure of a Pisum sativum L. population according to claim 9, wherein Pisum sativum L. germplasms are divided into two genetic subpopulations A and B according to the ΔK value.


SEQUENCE LISTING (184 sequences)

SEQ ID NO: 1 - DNA, 22 bp, Pisum sativum
  source 1..22: mol_type = other DNA
tcctaaacacagcactcaacac

SEQ ID NO: 2 - DNA, 20 bp, Pisum sativum
  source 1..20: mol_type = other DNA
accccaccagattgagatga

SEQ ID NO: 3 - DNA, 20 bp, Pisum sativum
  source 1..20: mol_type = other DNA
ccccaatcccctccaaaaca

SEQ ID NO: 4 - DNA, 20 bp, Pisum sativum
  source 1..20: mol_type = other DNA
gccgaagcctacttgtttgc

SEQ ID NO: 5 - DNA, 20 bp, Pisum sativum
  source 1..20: mol_type = other DNA
gccgaagcctacttgtttgc

SEQ ID NO: 6 - DNA, 20 bp, Pisum sativum
  source 1..20: mol_type = other DNA
gcatttcaagcatggggact

SEQ ID NO: 7 - DNA, 20 bp, Pisum sativum
  source 1..20: mol_type = other DNA
tgaggttgacgactgccttt

SEQ ID NO: 8 - DNA, 21 bp, Pisum sativum
  source 1..21: mol_type = other DNA
gcgcagcaccaaaataaggta

SEQ ID NO: 9 - DNA, 20 bp, Pisum sativum
  source 1..20: mol_type = other DNA
ccgaggcagagaaggaagac

SEQ ID NO: 10 - DNA, 22 bp, Pisum sativum
  source 1..22: mol_type = other DNA
tgtgcccttcataaccactgat

SEQ ID NO: 11 - DNA, 20 bp, Pisum sativum
  source 1..20: mol_type = other DNA
acgccattcctcgaacatct

SEQ ID NO: 12 - DNA, 20 bp, Pisum sativum
  source 1..20: mol_type = other DNA
ggcatgtctcctttgcaggt

SEQ ID NO: 13 - DNA, 23 bp, Pisum sativum
  source 1..23: mol_type = other DNA
ggttctgcatttccaaatcaact

SEQ ID NO: 14 - DNA, 22 bp, Pisum sativum
  source 1..22: mol_type = other DNA
tcacttccacctctttatccgc

SEQ ID NO: 15 - DNA, 20 bp, Pisum sativum
  source 1..20: mol_type = other DNA
tggttgggtgaggctgattt

SEQ ID NO: 16 - DNA, 20 bp, Pisum sativum
  source 1..20: mol_type = other DNA
ccgttgtagaggcgaagagt

SEQ ID NO: 17 - DNA, 20 bp, Pisum sativum
  source 1..20: mol_type = other DNA
tggtattgggtgctcttcgg

SEQ ID NO: 18 - DNA, 20 bp, Pisum sativum
  source 1..20: mol_type = other DNA
ataaatccgcctcccgaacc

SEQ ID NO: 19 - DNA, 22 bp, Pisum sativum
  source 1..22: mol_type = other DNA
ggttgtatttcactctcccgtt

SEQ ID NO: 20 - DNA, 21 bp, Pisum sativum
  source 1..21: mol_type = other DNA
atagaggggttgtgggtcaag

SEQ ID NO: 21 - DNA, 22 bp, Pisum sativum
  source 1..22: mol_type = other DNA
ttggattctttactgggagctg

SEQ ID NO: 22 - DNA, 20 bp, Pisum sativum
  source 1..20: mol_type = other DNA
tgggttggtggcagaatagg

SEQ ID NO: 23 - DNA, 20 bp, Pisum sativum
  source 1..20: mol_type = other DNA
gaaacagggaggtgtgggag

SEQ ID NO: 24 - DNA, 21 bp, Pisum sativum
  source 1..21: mol_type = other DNA
tggattgacgtggttgtttgg

SEQ ID NO: 25 - DNA, 21 bp, Pisum sativum
  source 1..21: mol_type = other DNA
ttatcgctagcaggggacaac

SEQ ID NO: 26 - DNA, 22 bp, Pisum sativum
  source 1..22: mol_type = other DNA
gataccgagatacagttcctgc

SEQ ID NO: 27 - DNA, 21 bp, Pisum sativum
  source 1..21: mol_type = other DNA
tcctcaattcccttcagtgcc

SEQ ID NO: 28 - DNA, 20 bp, Pisum sativum
  source 1..20: mol_type = other DNA
tggtaatggaggaggcgaga

SEQ ID NO: 29 - DNA, 20 bp, Pisum sativum
  source 1..20: mol_type = other DNA
gtctgaaaccgacaccgaca

SEQ ID NO: 30 - DNA, 20 bp, Pisum sativum
  source 1..20: mol_type = other DNA
ctgtccaaggaacagcctca

SEQ ID NO: 31 - DNA, 20 bp, Pisum sativum
  source 1..20: mol_type = other DNA
actcagggaaagtgtcgagc

SEQ ID NO: 32 - DNA, 20 bp, Pisum sativum
  source 1..20: mol_type = other DNA
tggaagaaacgaatggcgga

SEQ ID NO: 33 - DNA, 20 bp, Pisum sativum
  source 1..20: mol_type = other DNA
gccactgcatcccttgtttg

SEQ ID NO: 34 - DNA, 20 bp, Pisum sativum
  source 1..20: mol_type = other DNA
tcacaggaaactggggttcg

SEQ ID NO: 35 - DNA, 20 bp, Pisum sativum
  source 1..20: mol_type = other DNA
atgggatcagcttcggcttc

SEQ ID NO: 36 - DNA, 20 bp, Pisum sativum
  source 1..20: mol_type = other DNA
acaaagaagaagggcggtgg

SEQ ID NO: 37 - DNA, 24 bp, Pisum sativum
  source 1..24: mol_type = other DNA
tgaaccagtaacacaaaactacca

SEQ ID NO: 38 - DNA, 20 bp, Pisum sativum
  source 1..20: mol_type = other DNA
ctttggcagcaggagtgaga

SEQ ID NO: 39 - DNA, 19 bp, Pisum sativum
  source 1..19: mol_type = other DNA
gcctgacggaagaacggaa

SEQ ID NO: 40 - DNA, 21 bp, Pisum sativum
  source 1..21: mol_type = other DNA
aagaaacagcgagagccatga

SEQ ID NO: 41 - DNA, 20 bp, Pisum sativum
  source 1..20: mol_type = other DNA
gctaacagatgagccgaacg

SEQ ID NO: 42 - DNA, 20 bp, Pisum sativum
  source 1..20: mol_type = other DNA
accaatcaatgcctcctgcc

SEQ ID NO: 43 - DNA, 20 bp, Pisum sativum
  source 1..20: mol_type = other DNA
gagccgtttgattgctggtg

SEQ ID NO: 44 - DNA, 20 bp, Pisum sativum
  source 1..20: mol_type = other DNA
actgatcctgtggcaaacca

SEQ ID NO: 45 - DNA, 20 bp, Pisum sativum
  source 1..20: mol_type = other DNA
gaggcggaggaggaatagga

SEQ ID NO: 46 - DNA, 20 bp, Pisum sativum
  source 1..20: mol_type = other DNA
tttcagtggaaacccccaca

SEQ ID NO: 47 - DNA, 24 bp, Pisum sativum
  source 1..24: mol_type = other DNA
ccaacacaacacattagaagctca

SEQ ID NO: 48 - DNA, 21 bp, Pisum sativum
  source 1..21: mol_type = other DNA
tgctggttggttgtttggtag

SEQ ID NO: 49 - DNA, 22 bp, Pisum sativum
  source 1..22: mol_type = other DNA
cacattcacaagctcttcaggt

SEQ ID NO: 50 - DNA, 19 bp, Pisum sativum
  source 1..19: mol_type = other DNA
ggtggggaaccgatgagag

SEQ ID NO: 51 - DNA, 23 bp, Pisum sativum
  source 1..23: mol_type = other DNA
caacaaccacttccacagataca

SEQ ID NO: 52 - DNA, 22 bp, Pisum sativum
  source 1..22: mol_type = other DNA
ttccatccaactcacacatctc

SEQ ID NO: 53 - DNA, 21 bp, Pisum sativum
  source 1..21: mol_type = other DNA
acttcgatccgctatggacac

SEQ ID NO: 54 - DNA, 20 bp, Pisum sativum
  source 1..20: mol_type = other DNA
gtgcttcggggctatcatcg

SEQ ID NO: 55 - DNA, 20 bp, Pisum sativum
  source 1..20: mol_type = other DNA
tcttaccagcagagtcacgg

SEQ ID NO: 56 - DNA, 21 bp, Pisum sativum
  source 1..21: mol_type = other DNA
tctcaagctgcgaaagaggaa

SEQ ID NO: 57 - DNA, 20 bp, Pisum sativum
  source 1..20: mol_type = other DNA
agcctctaggtatccaggca

SEQ ID NO: 58 - DNA, 22 bp, Pisum sativum
  source 1..22: mol_type = other DNA
agagcttatcggcttacagcat

SEQ ID NO: 59 - DNA, 20 bp, Pisum sativum
  source 1..20: mol_type = other DNA
aacgcccgaaactcaaatgg

SEQ ID NO: 60 - DNA, 20 bp, Pisum sativum
  source 1..20: mol_type = other DNA
aggttgccagtgaaaggaga

SEQ ID NO: 61 - DNA, 20 bp, Pisum sativum
  source 1..20: mol_type = other DNA
agacagcaggtgttcgttgt

SEQ ID NO: 62 - DNA, 20 bp, Pisum sativum
  source 1..20: mol_type = other DNA
gcccctcaacgtgtctttgt

SEQ ID NO: 63 - DNA, 22 bp, Pisum sativum
  source 1..22: mol_type = other DNA
aagccttgacttgacgacatct

SEQ ID NO: 64 - DNA, 22 bp, Pisum sativum
  source 1..22: mol_type = other DNA
tgaatggttgaaggagaagggt

SEQ ID NO: 65 - DNA, 23 bp, Pisum sativum
  source 1..23: mol_type = other DNA
atatcaatctcggatagcagcac

SEQ ID NO: 66 - DNA, 20 bp, Pisum sativum
  source 1..20: mol_type = other DNA
ccgttccttcacagatgggt

SEQ ID NO: 67 - DNA, 20 bp, Pisum sativum
  source 1..20: mol_type = other DNA
ccaggcacagcaagagttga

SEQ ID NO: 68 - DNA, 21 bp, Pisum sativum
  source 1..21: mol_type = other DNA
caaactcgatttcaacgacgc

SEQ ID NO: 69 - DNA, 22 bp, Pisum sativum
  source 1..22: mol_type = other DNA
tccatgcacatttcctacacct

SEQ ID NO: 70 - DNA, 21 bp, Pisum sativum
  source 1..21: mol_type = other DNA
cccccttaagttggagagtga

SEQ ID NO: 71 - DNA, 20 bp, Pisum sativum
  source 1..20: mol_type = other DNA
ctgtcaaaaggctggaggca

SEQ ID NO: 72 - DNA, 23 bp, Pisum sativum
  source 1..23: mol_type = other DNA
acaaaagcgacaaccaaaaacga

SEQ ID NO: 73 - DNA, 20 bp, Pisum sativum
  source 1..20: mol_type = other DNA
tgttggtggttgtctgctca

SEQ ID NO: 74 - DNA, 20 bp, Pisum sativum
  source 1..20: mol_type = other DNA
gtttcgttcgctgccattgt

SEQ ID NO: 75 - DNA, 22 bp, Pisum sativum
  source 1..22: mol_type = other DNA
agcgaagaggatgacatgagta

SEQ ID NO: 76 - DNA, 20 bp, Pisum sativum
  source 1..20: mol_type = other DNA
tgcttcgtctgtttcgggag

SEQ ID NO: 77 - DNA, 20 bp, Pisum sativum
  source 1..20: mol_type = other DNA
gcgcatttacagtttgggct

SEQ ID NO: 78 - DNA, 20 bp, Pisum sativum
  source 1..20: mol_type = other DNA
cgacctcgagatgggaaacc

SEQ ID NO: 79 - DNA, 21 bp, Pisum sativum
  source 1..21: mol_type = other DNA
tgcgacgtaattgctcaaagt

SEQ ID NO: 80 - DNA, 21 bp, Pisum sativum
  source 1..21: mol_type = other DNA
aggctttcggaggaaaacaga

SEQ ID NO: 81 - DNA, 20 bp, Pisum sativum
  source 1..20: mol_type = other DNA
ggtgaacccttggcaacttc

SEQ ID NO: 82 - DNA, 20 bp, Pisum sativum
  source 1..20: mol_type = other DNA
atggtcgcttcccactttct

SEQ ID NO: 83 - DNA, 23 bp, Pisum sativum
  source 1..23: mol_type = other DNA
tggctgagaaagtgaaccttagt

SEQ ID NO: 84 - DNA, 19 bp, Pisum sativum
  source 1..19: mol_type = other DNA
tggtgtgtgtcggtggaaa

SEQ ID NO: 85 - DNA, 28 bp, Pisum sativum
  source 1..28: mol_type = other DNA
atagacaactagagattggtttttgaag

SEQ ID NO: 86 - DNA, 27 bp, Pisum sativum
  source 1..27: mol_type = other DNA
ggttaacaatgtcaatgtacacaatca

SEQ ID NO: 87 - DNA, 23 bp, Pisum sativum
  source 1..23: mol_type = other DNA
agggccagaagaagtaacaaaag

SEQ ID NO: 88 - DNA, 21 bp, Pisum sativum
  source 1..21: mol_type = other DNA
ttgggaaggatcagaagctgg

SEQ ID NO: 89 - DNA, 21 bp, Pisum sativum
  source 1..21: mol_type = other DNA
ctgtggaggcacaaatgaggt

SEQ ID NO: 90 - DNA, 20 bp, Pisum sativum
  source 1..20: mol_type = other DNA
cacgctcaacctcttcccat

SEQ ID NO: 91 - DNA, 21 bp, Pisum sativum
  source 1..21: mol_type = other DNA
acacgacggcagataaaagtg

SEQ ID NO: 92 - DNA, 20 bp, Pisum sativum
  source 1..20: mol_type = other DNA
gcgtttccgctgtttcctac

SEQ ID NO: 93 - DNA, 121 bp, synthetic construct
  source 1..121: mol_type = other DNA
ttttgcttgtccaatattgtattgtgagtgctggttttgaacctgaaattttttgtggtgrtcaacgattcagaaacatgaattattattttgtctttaacttgtatgtttgtcttaggat

SEQ ID NO: 94 - DNA, 121 bp, synthetic construct
  source 1..121: mol_type = other DNA
cttttcatcaaacgagttcatcgaatccaagcttgtgattctctacgcaaaatgcggcctygcggatatcgcggttcatctctttcgtaacgttgttcaaaaccagaatctcttctcttgg

SEQ ID NO: 95 - DNA, 121 bp, synthetic construct
  source 1..121: mol_type = other DNA
caccgagatggaacgttgaagcaaaaggttgcagagaatcccgagaggtatagatgtaaartcgctagtgtatgagttcgcgatgaaattatctgacttgccgtttttctttgaaatttaa

SEQ ID NO: 96 - DNA, 121 bp, synthetic construct
  source 1..121: mol_type = other DNA
tatatcttagaaccaatcatcagcaatgttgtttgaatatccttaactttagatttctgcygtcgtttgtccttattttgaatctgttgttgcaggctccaatggtggcctcatgtgaaag

SEQ ID NO: 97 - DNA, 121 bp, synthetic construct
  source 1..121: mol_type = other DNA
cttagtttctaattcatccacatgatgatttttgttagacaatttttgtttgagattctcyttattaagaaaatgcttagtcttaaacatggatcttcatggctattgttattttgtgaac

SEQ ID NO: 98 - DNA, 121 bp, synthetic construct
  source 1..121: mol_type = other DNA
aagcacattcaaaatttatttctcaaacatctcaaggcaattaggatcaaattcaggcackgaagtaccgaaacaaacctgaactgcagctctatagaagagttcctctcccactgaactt

SEQ ID NO: 99 - DNA, 121 bp, synthetic construct
  source 1..121: mol_type = other DNA
tgtacaatcattataaatatagctattgtgaagagatgttatgcggaagaaaagaaaaackaagctatgattttattctacaacctctttcttgctcaaacaaatagaactagagcaagat

SEQ ID NO: 100 - DNA, 121 bp, synthetic construct
  source 1..121: mol_type = other DNA
ggacaagaaatcagttttgatgatttatctcttgaagagaagaaacaatttcaaagagctrttgcttgtggggaattaagcaagatgatcacaccatgggatccatggtggtcaaagcatt

SEQ ID NO: 101 - DNA, 121 bp, synthetic construct
  source 1..121: mol_type = other DNA
agttagactttggctaattggcattgcttctgtcttttttctgtttcagtttccaattgcrcttttggtattcggctttgaagcagtaaagttgtacaaggatcacagaaagaggaagggc

SEQ ID NO: 102 - DNA, 121 bp, synthetic construct
  source 1..121: mol_type = other DNA
acgtcaaggcagcgagagatatggtgtcatccgaaagccgaaactctacgagaggtgaaayttcgttgaagtaaacttctaccttaatcaaaaattgaggaagaaatattttttgagatgt

SEQ ID NO: 103 - DNA, 121 bp, synthetic construct
  source 1..121: mol_type = other DNA
aactttcagggcgaggtgattcataactggaactttcagaggtaggtgattcatacaaacyggcactttcagagctttcaaaatcaggaacttcgccatcaaaggaagtatcaggagcatc

SEQ ID NO: 104 - DNA, 121 bp, synthetic construct
  source 1..121: mol_type = other DNA
catcatgtgctcaactttggtcacattttccacgtttctcaggtgctaagaagttcaaaaycaaatagtcttccctccatctcctcttccgcgccgaaggtcgcctctggcggcggcggac

SEQ ID NO: 105 - DNA, 121 bp, synthetic construct
  source 1..121: mol_type = other DNA
cgtattttggtaacacaccaccaaatcagagagtctcgatgctgagattccggaatcaacrcacgctgtttgtagatctttgcataagaaacgtcgaggatttaactcgaaataatcgaat

SEQ ID NO: 106 - DNA, 121 bp, synthetic construct
  source 1..121: mol_type = other DNA
aaatccacattaggtctctctactttcatgtccatatccaaatcagaacccctcccctcayacacattgtccttattcaaccaagatctttcatccaaaccttcatactggcaagaaacat

SEQ ID NO: 107 - DNA, 121 bp, synthetic construct
  source 1..121: mol_type = other DNA
gaggaattatggagagacaatgagacatggagaaagacagctacaaaacaaaaaacaaaartgcaattgaacttcactatgatactgtttttgtttcttacatggttgtctgtttttgtgt

SEQ ID NO: 108 - DNA, 121 bp, synthetic construct
  source 1..121: mol_type = other DNA
ttcttcttcttcaagctgcttaagccaaagaatggcatgaaccctctgtctcataatcctrtaatccttagcaagcttctcaacagtataaacctcaggatcctttttgtgcaagcgatac

SEQ ID NO: 109 - DNA, 121 bp, synthetic construct
  source 1..121: mol_type = other DNA
gcttccagaatagcttgattagcggtgaagtgagcgaatttgagataaggagaagactcgkaaaagtgcatgtgaagtgcatcagacaaagatgaatccagagtttcatcaggggaaactc

SEQ ID NO: 110 - DNA, 121 bp, synthetic construct
  source 1..121: mol_type = other DNA
agccactaattgacaattagacatagttgtatatgtatatatgttagtgaggacaataagraaagaaaatcagattgtatatacattaagcgtggtgaagaaatatatgtgaaaaagcctt

SEQ ID NO: 111 - DNA, 121 bp, synthetic construct
  source 1..121: mol_type = other DNA
ctaatttaatcaactttcattcaaaagcaaaacaccttgcagcatagcacaactgcaagamgagcacaatggcactatatacataagagaggttttaatcaccctaaacaaccaaaaaaga

SEQ ID NO: 112 - DNA, 121 bp, synthetic construct
  source 1..121: mol_type = other DNA
agaatgcaatctgacgaaagccatgaggcacgaagtttataccatcatccactaaaaacaygccacgtaatcatgccaaaactccgtccaggttcaataaaccgccacaatagagaaacct

SEQ ID NO: 113 - DNA, 121 bp, synthetic construct
  source 1..121: mol_type = other DNA
catggatcctctggagtctttcttcgcgtcctgactttttaacgagtgaaatagttggacrgttgcagaagccttatgagtgttggccaattttgccaaaggccgagacggaatcggcggt

SEQ ID NO: 114 - DNA, 121 bp, synthetic construct
  source 1..121: mol_type = other DNA
ctgatacaccttaccctccgcaaaagtcaaacaaagatgcttcccagaaagaaaatgttartgaatatgcaaaacatgacatcataagtggtgaaagcaacagtgtggtgtcactgaaaca

SEQ ID NO: 115 - DNA, 121 bp, synthetic construct
  source 1..121: mol_type = other DNA
catgtcataggggcctagaccgtaacccatcatatgcattggaccagcagcagcatatggkgccataaatccatccataccaggttggataccattccagtatgggttataaccaggaggt

SEQ ID NO: 116 - DNA, 121 bp, synthetic construct
  source 1..121: mol_type = other DNA
acagaaagtcaagatcgcagaaaagatattcccgatgataactgagacgacgaggacaccracccatttgaccctctcaacgaaattatcaaaaaattccagtaaagagaattgatttgcc

SEQ ID NO: 117 - DNA, 121 bp, synthetic construct
  source 1..121: mol_type = other DNA
ttgagaacacttgcaatttcaacaatcacatgataatctctggaaacattcttaagagctrcatttgccaacacagcataacaatctgaaggttccaatatccacctatacattgctttct

SEQ ID NO: 118 - DNA, 121 bp, synthetic construct
  source 1..121: mol_type = other DNA
tttaatcttatatatacatacatgaaggaaaaaaaatatagaatcaaaaaattgtaccatyagaagaagcagataaggttggaatttgtgagcttggaaaccaagcaaagaattatcctaa

SEQ ID NO: 119 - DNA, 121 bp, synthetic construct
  source 1..121: mol_type = other DNA
cacagcgcaacgaccgtgggacccacctctcgttcaagagggtcgggttcggtccttctgyacgggtcggaagtttcgcaatcttttcaaatatccgcttcatcgtgtcttcgtttcaccg

SEQ ID NO: 120 - DNA, 121 bp, synthetic construct
  source 1..121: mol_type = other DNA
ggaatcatgacaggttcgatggaaccgtatttggaaaaactgagacactatgcaggtgtgrtacctttgttgaccgctgattacggagcctctgaaggatggatagctgcaaatgtgaatc

SEQ ID NO: 121 - DNA, 121 bp, synthetic construct
  source 1..121: mol_type = other DNA
aaaaaaataacagactcaaactctatccaatgggatattaaagcaatacgcaaccaaagayggaagcaggcacaaacgaccagcatttttggaaagtgcactcttgtggcaaatgattgcg

SEQ ID NO: 122 - DNA, 121 bp, synthetic construct
  source 1..121: mol_type = other DNA
atttccttatttatgtgtcaaaagatcgatcgcgcaaaataaaataaaatacaaatctccrccgatacaatgtattttcttttccacaaacaaaacaaaacaaaagttaccattttcttct

SEQ ID NO: 123 - DNA, 121 bp, synthetic construct
  source 1..121: mol_type = other DNA
aatcatcaacaaaaatacatagcgagacttctctatactcttttatgattttcgaatatgraccataactacaaaataaaccacaagtggcaatgcagtaatgcataaatacctgtttgac

SEQ ID NO: 124 - DNA, 121 bp, synthetic construct
  source 1..121: mol_type = other DNA
ttttggagccaaggatgctctgcaagacaagtaaaggaaaacatcatgaagtcagtgaatytattctgataaatccttaacataaacagcataaatgatttctaatcattatttatttcag

SEQ ID NO: 125 - DNA, 121 bp, synthetic construct
  source 1..121: mol_type = other DNA
agcatggccatccccaaaaagggaacgagagctgagagcagaacggccgataattagtgaygcggacactaaaacattatcaaaattcatgaagtgttcatattaagaaataaaaatcata

SEQ ID NO: 126 - DNA, 121 bp, synthetic construct
  source 1..121: mol_type = other DNA
gtgtttgaagttccgattgaaaagttggaaatcgaattcagaaagcctgtagatcagttaragaaggaaacaatatcggagtcagagaagccttttgtgaacgaactgacaatgaaaaacg

SEQ ID NO: 127 - DNA, 121 bp, synthetic construct
  source 1..121: mol_type = other DNA
tttgttttccacgaaagaactaaacacattaaagtggattgtcatttcataagagaccatrtttaatcaggtgtcatcactatgactcatgtttcaactcatcagcaataggctgacatct

SEQ ID NO: 128 - DNA, 121 bp, synthetic construct
  source 1..121: mol_type = other DNA
ggtttatagtcgtccaaaaactaaggtagcggtagtaccgtcagaaatagagcagaagagyggtgaaagtgatggtgaatatattaagcggctgaaagcttttgttgagaatcagcactct

SEQ ID NO: 129 - DNA, 121 bp, synthetic construct
  source 1..121: mol_type = other DNA
acaagcacaggaaaggttactaatcatacctatttctaagtgtggcatagttagaatgcaraaagagtagtacgatcaaaagcagcaagacatcagaacaaattgttaagaatggtaaatc

SEQ ID NO: 130 - DNA, 121 bp, synthetic construct
  source 1..121: mol_type = other DNA
ggaacattttgtctgtctaggacctttgtagcaccgacacgtctgaatttttttttggaaygattggattgaatgacttaatcagagtatgaggatcattcctgtagtaccttttttcaac

SEQ ID NO: 131 - DNA, 121 bp, synthetic construct
  source 1..121: mol_type = other DNA
tgaaggagttggacaattattcgttggtgtcgaatacgagggaatgacttgaggagctttrcagtaaacctcattagaaactccattttcaagacaaatcttgttttgacccaatctgcaa

SEQ ID NO: 132 - DNA, 121 bp, synthetic construct
  source 1..121: mol_type = other DNA
atgtaaagactatggaagagcttacctgatcaattttacttcgaaaaatacgtttcaagakcctgttaaagtgtttgttttggtgaatcttcccaatttcgtcgtgcagtttttctagcaa

SEQ ID NO: 133 - DNA, 121 bp, synthetic construct
  source 1..121: mol_type = other DNA
aactccttcctaagttctgagaaagtttaagttatgaaaaatataatgtttaaggccttartgatgacaattacccaataataagataagttgaatgcatatgatgcaggtactctgacgg

SEQ ID NO: 134 - DNA, 121 bp, synthetic construct
  source 1..121: mol_type = other DNA
atctgtaaattcagcaacacacattgttggaggcaaggttggttggaatttgcctagttaytatagtttctttgaggattggtcaaagaaccaaaccttcattgttggcgatcaacttcgt

SEQ ID NO: 135 - DNA, 121 bp, synthetic construct
  source 1..121: mol_type = other DNA
tgttgtcactatcaaagacaaatttgatgatagtgagataggaaaggaataaaacacagtrtttagatcatagataatatttgtacacttaaagttttaacatttgtgacacctagttcaa

SEQ ID NO: 136 - DNA, 121 bp, synthetic construct
  source 1..121: mol_type = other DNA
caaatgacccattcaatcattttctgttggccatgttgcctaagccttgtttggcagtgtygggatactttgttcattccagaaaaagttttgaggatcaacttgagtcttaaccttattt

SEQ ID NO: 137 - DNA, 121 bp, synthetic construct
  source 1..121: mol_type = other DNA
tctaaaatacattcctgatggaaactacataaaggttggaagcgtcgccacaatcaacaarccagacttgttgccaacactctccacattgcgttactttaccaacacgttatctaaaaaa

SEQ ID NO: 138 - DNA, 121 bp, synthetic construct
  source 1..121: mol_type = other DNA
cctgttcttcacattgaatcccttggtcatgcccttcatgcttttataaacgggaaactcrcaggtatctttctttacttcaatgaagaatctcatgaatcatgtaatgatatatcgatat

SEQ ID NO: 139 - DNA, 30 bp, synthetic construct
  source 1..30: mol_type = other DNA
ttttttttttacctgaaattttttgtggtg

SEQ ID NO: 140 - DNA, 35 bp, synthetic construct
  source 1..35: mol_type = other DNA
tttttttttttttttttttacgcaaaatgcggcct

SEQ ID NO: 141 - DNA, 40 bp, synthetic construct
  source 1..40: mol_type = other DNA
tttttttttttttttttttcccgagaggtatagatgtaaa

SEQ ID NO: 142 - DNA, 53 bp, synthetic construct
  source 1..53: mol_type = other DNA
tttttttttttttttttttttttttttttatatccttaactttagatttctgc

SEQ ID NO: 143 - DNA, 57 bp, synthetic construct
  source 1..57: mol_type = other DNA
ttttttttttttttttttttttttttttttttttaagactaagcattttcttaataa

SEQ ID NO: 144 - DNA, 61 bp, synthetic construct
  source 1..61: mol_type = other DNA
ttttttttttttttttttttttttttttttttttttttttattaggatcaaattcaggcac

SEQ ID NO: 145 - DNA, 65 bp, synthetic construct
  source 1..65: mol_type = other DNA
ttttttttttttttttttttttttttttttttttttttttaggttgtagaataaaatcatagctt

SEQ ID NO: 146 - DNA, 49 bp, synthetic construct
  source 1..49: mol_type = other DNA
ttttttttttttttttttttttttttagaagaaacaatttcaaagagct

SEQ ID NO: 147 - DNA, 30 bp, synthetic construct
  source 1..30: mol_type = other DNA
ttttttttttctgtttcagtttccaattgc

SEQ ID NO: 148 - DNA, 38 bp, synthetic construct
  source 1..38: mol_type = other DNA
ttttttttttttttttcgaaactctacgagaggtgaaa

SEQ ID NO: 149 - DNA, 45 bp, synthetic construct
  source 1..45: mol_type = other DNA
tttttttttttttttttttttcagaggtaggtgattcatacaaac

SEQ ID NO: 150 - DNA, 53 bp, synthetic construct
  source 1..53: mol_type = other DNA
ttttttttttttttttttttttttttttttttcaggtgctaagaagttcaaaa

SEQ ID NO: 151 - DNA, 40 bp, synthetic construct
  source 1..40: mol_type = other DNA
tttttttttttttttttttttctgagattccggaatcaac

SEQ ID NO: 152 - DNA, 61 bp, synthetic construct
  source 1..61: mol_type = other DNA
ttttttttttttttttttttttttttttttttttttttttttttcagaacccctcccctca

SEQ ID NO: 153 - DNA, 65 bp, synthetic construct
  source 1..65: mol_type = other DNA
ttttttttttttttttttttttttttttttttttttttttagtatcatagtgaagttcaattgca

SEQ ID NO: 154 - DNA, 57 bp, synthetic construct
  source 1..57: mol_type = other DNA
tttttttttttttttttttttttttttttttttttgaaccctctgtctcataatcct

SEQ ID NO: 155 - DNA, 30 bp, synthetic construct
  source 1..30: mol_type = other DNA
tttttttttttgagataaggagaagactcg

SEQ ID NO: 156 - DNA, 42 bp, synthetic construct
  source 1..42: mol_type = other DNA
ttttttttttttttttgtatatatgttagtgaggacaataag

SEQ ID NO: 157 - DNA, 35 bp, synthetic construct
  source 1..35: mol_type = other DNA
tttttttttttttttagcatagcacaactgcaaga

SEQ ID NO: 158 - DNA, 49 bp, synthetic construct
  source 1..49: mol_type = other DNA
tttttttttttttttttttttttttttataccatcatccactaaaaaca

SEQ ID NO: 159 - DNA, 61 bp, synthetic construct
  source 1..61: mol_type = other DNA
tttttttttttttttttttttttttttttttttttttttttaacgagtgaaatagttggac

SEQ ID NO: 160 - DNA, 57 bp, synthetic construct
  source 1..57: mol_type = other DNA
tttttttttttttttttttttttttttttttttttgatgtcatgttttgcatattca

SEQ ID NO: 161 - DNA, 40 bp, synthetic construct
  source 1..40: mol_type = other DNA
ttttttttttttttttttttttaccagcagcagcatatgg

SEQ ID NO: 162 - DNA, 65 bp, synthetic construct
  source 1..65: mol_type = other DNA
ttttttttttttttttttttttttttttttttttttttttttttttctgagacgacgaggacacc

SEQ ID NO: 163 - DNA, 30 bp, synthetic construct
  source 1..30: mol_type = other DNA
tttttttctctggaaacattcttaagagct

SEQ ID NO: 164 - DNA, 61 bp, synthetic construct
  source 1..61: mol_type = other DNA
ttttttttttttttttttttttttttttttttttttttttccaaccttatctgcttcttct

SEQ ID NO: 165 - DNA, 35 bp, synthetic construct
  source 1..35: mol_type = other DNA
ttttttttttttttttttcgggttcggtccttctg

SEQ ID NO: 166 - DNA, 40 bp, synthetic construct
  source 1..40: mol_type = other DNA
ttttttttttttttttttactgagacactatgcaggtgtg

SEQ ID NO: 167 - DNA, 45 bp, synthetic construct
  source 1..45: mol_type = other DNA
ttttttttttttttttttttttttttagcaatacgcaaccaaaga

SEQ ID NO: 168 - DNA, 52 bp, synthetic construct
  source 1..52: mol_type = other DNA
ttttttttttttttttttttttttttcaaaataaaataaaatacaaatctcc

SEQ ID NO: 169 - DNA, 57 bp, synthetic construct
  source 1..57: mol_type = other DNA
tttttttttttttttttttttttttttttttatactcttttatgattttcgaatatg

SEQ ID NO: 170 - DNA, 53 bp, synthetic construct
  source 1..53: mol_type = other DNA
tttttttttttttttttttttttttttttgaaaacatcatgaagtcagtgaat

SEQ ID NO: 171 - DNA, 40 bp, synthetic construct
  source 1..40: mol_type = other DNA
ttttttttttttttttttttgaacggccgataattagtga

SEQ ID NO: 172 - DNA, 35 bp, synthetic construct
  source 1..35: mol_type = other DNA
tttttttttttttcagaaagcctgtagatcagtta

SEQ ID NO: 173 - DNA, 45 bp, synthetic construct
  source 1..45: mol_type = other DNA
tttttttttttttttttttttgattgtcatttcataagagaccat

SEQ ID NO: 174 - DNA, 49 bp, synthetic construct
  source 1..49: mol_type = other DNA
tttttttttttttttttttttttttttcgtcagaaatagagcagaagag

SEQ ID NO: 175 - DNA, 53 bp, synthetic construct
  source 1..53: mol_type = other DNA
tttttttttttttttttttttttttttttttagtgtggcatagttagaatgca

SEQ ID NO: 176 - DNA, 57 bp, synthetic construct
  source 1..57: mol_type = other DNA
ttttttttttttttttttttttttttttttttttttcgtctgaatttttttttggaa

SEQ ID NO: 177 - DNA, 61 bp, synthetic construct
  source 1..61: mol_type = other DNA
tttttttttttttttttttttttttttttttttttttttttggaatgacttgaggagcttt

SEQ ID NO: 178 - DNA, 55 bp, synthetic construct
  source 1..55: mol_type = other DNA
tttttttttttttttttttttttttttttttttcttcgaaaaatacgtttcaaga

SEQ ID NO: 179 - DNA, 29 bp, synthetic construct
  source 1..29: mol_type = other DNA
tttttcttattattgggtaattgtcatca

SEQ ID NO: 180 - DNA, 39 bp, synthetic construct
  source 1..39: mol_type = other DNA
tttttttttttttttttttggttggaatttgcctagtta

SEQ ID NO: 181 - DNA, 40 bp, synthetic construct
  source 1..40: mol_type = other DNA
ttttttttttttttttgataggaaaggaataaaacacagt

SEQ ID NO: 182 - DNA, 45 bp, synthetic construct
  source 1..45: mol_type = other DNA
ttttttttttttttttttttttttttaagccttgtttggcagtgt

SEQ ID NO: 183 - DNA, 50 bp, synthetic construct
  source 1..50: mol_type = other DNA
ttttttttttttttttttttttttttttttttcgtcgccacaatcaacaa

SEQ ID NO: 184 - DNA, 60 bp, synthetic construct
  source 1..60: mol_type = other DNA
tttttttttttttttttttttttttttttttttttttttgcttttataaacgggaaactc